(19) United States
(12) Patent Application Publication
(10) Pub. No.: US 2026/0041703 A1
(43) Pub. Date: Feb. 12, 2026

(54) HYPERBRANCHED DEXTRINS FOR TOPICAL USE IN THE PREVENTION OR TREATMENT OF AT LEAST ONE SYMPTOM OF SKIN INFLAMMATION

(71) Applicant: ROQUETTE FRERES, Lestrem (FR)

(72) Inventors: Géraldine LOUVET-POMMIER, Lachelle (FR); Léon MENTINK, LILLE (FR); Daniel WILS, MORBECQUE (FR)

(21) Appl. No.: 18/852,831

(22) PCT Filed: Mar. 30, 2023
(86) PCT No.: PCT/EP2023/025145

§ 371 Date: May 21, 2025

(30) Foreign Application Priority Data
Mar. 30, 2022 (FR) FR2202905

(51) Int. Cl. A61K 31/718; A61K 9/00; A61P 17/16

(52) U.S. Cl. CPC A61K 31/718; A61K 9/0014; A61P 17/16

(57) ABSTRACT

Hyperbranched dextrins, preferentially hyperbranched and hydrogenated dextrins, for topical use in the prevention or treatment of at least one symptom selected from redness, heat, swelling and pain, as well as in the prevention or treatment of the symptoms of skin inflammation and of mucosal inflammation. The invention also relates to the use of these hyperbranched, and preferably hyperbranched and hydrogenated, dextrins to prevent or reduce or eliminate the irritant effect induced by ingredients contained in cosmetic or dermatological compositions.

### TECHNICAL FIELD

The present invention relates to hyperbranched dextrins, preferentially hyperbranched and hydrogenated dextrins, for topical use in the prevention or treatment of at least one symptom selected from redness, heat, swelling and pain, as well as in the prevention or treatment of the symptoms of skin inflammation and of mucosal inflammation. The invention also relates to the use of these hyperbranched, and preferentially hyperbranched and hydrogenated, dextrins to prevent or reduce or eliminate the irritant effect induced by ingredients contained in cosmetic, dermatological or other compositions.

### PRIOR ART

The skin is both a living anatomical barrier and a zone of exchange between the body and its environment, the effectiveness of which determines the maintenance of a good homeostatic balance. It comprises a superficial layer consisting of the epidermis, and deeper layers forming the dermis and hypodermis. Each of these layers has specific properties allowing the skin to react and adapt to the conditions of its environment.

The mucous membranes forming the object of this application are the membranes lining the internal cavities of the body, selected from the mouth and genitals. This lining is a stack of cells called epithelium. This epithelium contains specific cells that produce mucus, hence their name. This mucus lubricates and protects the organ lined therewith. Mucous membranes play a protective role and are connected to the skin. Inflammation of the mucosa, mucosal folds or mucosal lesions may thus sometimes be observed.

As the protective barrier of the body, the skin and mucous membranes are exposed to a wide range of aggressions which can lead to discomfort or even, in the case of very intense or more severe reactions, inflammation of the skin or mucous membranes.

Skin inflammation can notably be induced by irritation of the skin which occurs when the skin comes into contact with irritant agents, such as chemical substances like cleansers, or such as mechanical actions like shaving, scrubbing, peels or hair removal. Skin irritation may also be caused by the action of the temperature, weather, ultra-violet radiation or even air pollution.

Irritation of the mucous membranes, for example the buccal mucosa may also be induced by certain foods or drinks, leading to the formation of mouth ulcers, for example, or by friction, such as brushing with a toothbrush. The genital mucosa may also be irritated locally, following repeated rubbing, over-aggressive soap, too many eggs, very frequent intercourse on slightly dry mucosa or imbalanced flora.

These irritants cause irritation of the skin or mucous membranes, activating the innate immune response, the first stage of which is inflammation of the irritated tissue or mucous membranes. Inflammation of the skin or mucous membranes may also be caused by pathologies involving an inflammatory process.

The action of an irritant chemical substance depends on its ability to penetrate the upper layer of the skin, the stratum corneum, which acts as a protective barrier. As far as mucous membranes are concerned, penetration of an irritant chemical substance is much easier because mucous membranes do not have a physical barrier made of keratin cells, but are simply made of non-keratinized epithelial cells into which the substance can easily penetrate. Once the skin barrier has been breached, or once the irritant has penetrated the mucous membrane, the irritant then comes into contact with living cells and interacts with the endogenous substances naturally present therein, thus disrupting the biological functioning of skin or mucous membrane cells, or even causing tissue damage. The first biological response is an acute inflammatory reaction in the cells of the damaged tissue. This reflects the cells' defense against the invasive product in order to accelerate the elimination thereof based on the innate immune response.

Irritating substances cause reversible skin damage. By applying them directly to the epidermal surface, their irritant potential can be assessed through various parameters such as the expression levels of the main biomarkers playing a role in skin inflammation.

The inflammatory phenomenon is made up of four symptoms: pain, heat, redness and swelling of the tissue. Its intensity is proportional to the degree of irritation (dose-dependent response) and to the amount of cytokines produced by epidermal keratinocytes to induce and control the early inflammatory response. Cytokines having a major role in the inflammatory cellular response are interleukins IL-1α, IL-6 and IL-8 and tumor necrosis factor α (TNF-α). IL-1β, IL-8, RANTES and VEGF are also biomarkers of inflammation.

There is an ongoing need for new agents, of natural or plant origin, capable of preventing or effectively treating a symptom selected from redness, heat, pain and swelling, as well as preventing or effectively treating inflammation of the skin or mucous membranes.

In addition, compounds known to irritate the skin such as oxidizing products for example used for dyeing hair, abrasive products such as synthetic micro-plastics, mineral powders or pigments in cosmetic creams, surfactants and solvents used in washing or cleansing compositions, acids such as salicylic acid used in peels, compositions for exfoliating the skin or promoting cell renewal, or else retinoids used in anti-aging compositions, are generally used in low doses. However, even small quantities of these compounds can cause irritation to sensitive skin.

There is thus also a need to identify compounds that can prevent or reduce or eliminate the irritant effect of ingredients, in particular ingredients contained in topical compositions for cosmetic, dermocosmetic, dermatological, pharmaceutical, medicinal, veterinary or domestic use.

### SUMMARY

The applicant has demonstrated, using a two-dimensional reconstructed human epidermis model, that hyperbranched hydrogenated dextrins according to the invention induce a decrease in 4 biomarkers involved in inflammation (IL-1α, IL-1β, RANTES and VEGF).

The assay of the inflammatory markers IL-1α and IL-8 (following inflammation by sodium lauryl sulfate (SLS) in 0.5% aqueous solution for 6 hours showed an action profile of hyperbranched hydrogenated dextrins similar to that of Betamethasone 0.05%, the reference positive control for anti-inflammatory effect.

Following these results, further research revealed additional biomarkers of inflammation, also impacted by the treatments performed.

In this subsequent study, the IL-1α assay confirmed the previously obtained results. In fact, the overexpression of IL-1α during treatment with SLS was compensated for by treatment with hyperbranched hydrogenated dextrins, bringing it back below the threshold value for interleukin overexpression. A decrease in RANTES and VEGF expression was also observed by treatment with hyperbranched hydrogenated dextrins after SLS inflammation.

Furthermore, IL-1β has also been shown to be under-expressed in the presence of hyperbranched hydrogenated dextrin treatment.

Thus, it was found that, for these 4 biomarkers involved in the mechanism of inflammation, treatment with hyperbranched hydrogenated dextrins induced a decrease in these signals.

The applicant has also demonstrated that hyperbranched dextrins according to the invention maintain the integrity of reconstructed human epidermal cell membranes as effectively as betamethasone.

It has also been demonstrated by the applicant that the application of an aftershave lotion comprising hyperbranched and hydrogenated dextrins according to the invention, reduced intracellular water loss compared to the placebo lotion.

Finally, the applicant has shown that dextrins according to the invention significantly reduce SLS-induced redness as early as 10 minutes compared with the placebo and reference groups, with this reduction being maintained for up to 7 days thereafter. It has also been shown that dextrins according to the invention significantly reduce transepidermal water loss as early as 30 minutes after application, then progressively increased over the treatment period from 2 to 7 days, to reach a water loss reduction value greater than that of the placebo group by around 6.6 units (that is, a difference of around 195%). Dextrins according to the invention therefore help maintain skin hydration by maintaining the barrier function of the skin.

Thus, the present invention relates to hyperbranched dextrins, preferentially hyperbranched and hydrogenated dextrins for topical use for the prevention or treatment of at least one symptom selected from redness, heat, swelling and pain.

The present invention also relates to the use of hyperbranched dextrins, preferentially hyperbranched and hydrogenated dextrins, to prevent or reduce or eliminate the irritant effect of ingredients contained in topical compositions for cosmetic, dermocosmetic, dermatological, pharmaceutical, medicinal, veterinary or domestic use (for example detergents).

### DETAILED DESCRIPTION

### Hyperbranched Dextrin

In a first embodiment, the dextrins for therapeutic use forming the object of the present application are hyperbranched dextrins.

By “dextrin”, the applicant means glucose polymers obtained from granular starch by pyroconversion, usually by the action of an acid in a generally dry environment, in other words, on granules in the form of dry solid particles, containing residual moisture imposed by physical equilibrium conditions at a given temperature and pressure. Thus, maltodextrins and pyrogenic dextrins fall into the general family of dextrins useful for the invention when they have been modified to be hyperbranched.

In the context of the invention, by “dextrin”, the applicant also means glucose polymers derived from the acid or enzymatic liquefaction or hydrolysis of starch and generally referred to as “maltodextrin”, “starch hydrolysate” or “glucose syrup”, when these polymers have been further chemically or enzymatically modified, in particular by branching enzymes, to present glucosidic bonds between the anhydroglucose molecules, significantly different in nature from those of the starch from which they are derived. Thus, maltodextrins, starch hydrolysates, glucose syrups and pyrogenic dextrins fall into the general family of dextrins useful for the invention as they differ in terms of glucosidic bonds from the starch from which they are derived, by comprising “atypical” bonds.

By “hyperbranched dextrin”, the applicant means a “dextrin” having glucosidic bonds between the anhydroglucose molecules, significantly different in nature and quantity from those naturally constituting the starch from which it is derived. It comprises glucosidic bonds between anhydroglucose molecules that are naturally present, 1,6 bonds, but in greater quantity, and glucosidic bonds not naturally present in starch, known as “atypical”, 1,3 and 1,2 bonds. Thus a hyperbranched dextrin is a dextrin comprising a large proportion of branching glucosidic bonds relative to the total glucosidic bonds present in said dextrin. The term “hyperbranched” may also be replaced by the term “highly branched”.

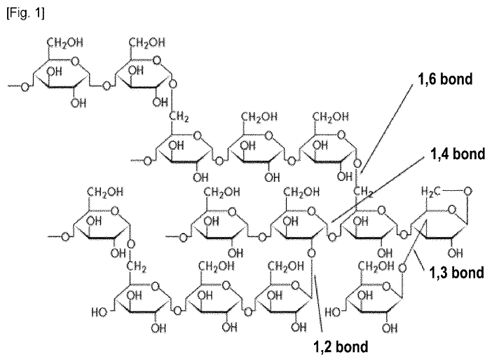

“Large proportion” is understood to mean a proportion of branching glucosidic bonds greater than or equal to 5% of the sum of branching glucosidic bonds and linear glucosidic bonds, preferentially greater than or equal to 10%, more preferentially greater than or equal to 20%, more preferentially greater than or equal to 30%, more preferentially greater than or equal to 40%, more preferentially greater than or equal to 50%. Glucosidic branching bonds are bonds that generate non-linear chains, in higher proportions than the normal values for native starch. The glucosidic bonds that generate non-linear chains are 1,6, 1,3, and 1,2 glucosidic bonds, as shown in FIG. 1 [FIG. 1]. The 1,3 and 1,2 bonds are atypical glucosidic bonds. Normal values for these bonds in native starch are around 5% for 1,6 bonds, and around 0% for 1,2 and 1,3 bonds. The complement is made up of 1,4 glucosidic bonds, that is, around 95%. The 1,4 bond is a bond that creates a linear bond.

For 1,6 glucosidic bonds, characteristic values of hyperbranched dextrins useful for the invention are values greater than 5% relative to the sum of 1,2, 1,3, 1,4 and 1,6 bonds, preferentially greater than or equal to 10%, relative to the sum of 1,2, 1,3, 1,4 and 1,6 bonds, and more preferentially greater than or equal to 12% relative to the sum of 1,2, 1,3, 1,4 and 1,6 bonds, and most preferentially greater than or equal to 15% relative to the sum of 1,2, 1,3, 1,4 and 1,6 bonds.

Thus, according to one embodiment, hyperbranched dextrins comprise at least 5%, preferentially at least 10%, preferentially at least 12%, preferentially at least 15% of 1,6 glucosidic bonds relative to the sum of 1,2, 1,3, 1,4 and 1,6 bonds.

For 1,2 and 1,3 glucosidic bonds, values useful for the invention are values greater than 1% relative to the sum of 1,2, 1,3, 1,4 and 1,6 bonds, preferentially greater than or equal to 5% relative to the sum of 1,2, 1,3, 1,4 and 1,6 bonds, and more preferentially greater than or equal to 10% relative to the sum of 1,2, 1,3, 1,4 and 1,6 bonds.

Thus, according to one embodiment, hyperbranched dextrins comprise at least 1%, preferentially at least 5%, preferentially at least 10%, and preferentially at least 20% of 1,2 glucosidic bonds relative to the sum of 1,2, 1,3, 1,4 and 1,6 bonds.

According to one embodiment, hyperbranched dextrins comprise at least 1%, preferentially at least 5%, preferentially at least 10% and preferentially at least 20% of 1,3 glucosidic bonds relative to the sum of 1,2, 1,3, 1,4 and 1,6 bonds.

For 1,4 glucosidic bonds, values useful for the invention are values less than 90%, preferentially less than 70%, preferentially less than 60%, preferentially less than 50%, relative to the sum of 1,2, 1,3, 1,4 and 1,6 bonds, and preferentially between 42 and 50%, relative to the sum of 1,2, 1,3, 1,4 and 1,6 bonds.

Thus, according to one embodiment, branched dextrins comprise at most 90%, preferentially at most 70%, preferentially at most 50% of 1,4 glucosidic bonds relative to the sum of 1,2, 1,3, 1,4 and 1,6 bonds.

Preferentially, hyperbranched, preferably hyperbranched and hydrogenated dextrins, useful for the invention have a 1,6 glucosidic bond content of between 5% and 40%, preferentially between 10% and 30%, more preferentially between 12% and 22%, and most preferentially between 15 and 20%, relative to the sum of 1,2, 1,3, 1,4 and 1,6 bonds.

According to one embodiment, the hyperbranched, preferably hyperbranched and hydrogenated dextrins, useful for the invention comprise:
at most 90% of 1,4 glucosidic bonds,
at least 5% of 1,6 glucosidic bonds,
   relative to the sum of 1,2, 1,3, 1,4 and 1,6 bonds.

According to one embodiment, the hyperbranched, preferably hyperbranched and hydrogenated dextrins, useful for the invention comprise:
at most 70% of 1,4 glucosidic bonds,
at least 5% of 1,6 glucosidic bonds,
   relative to the sum of 1,2, 1,3, 1,4 and 1,6 bonds.

According to one embodiment, hyperbranched, preferably hyperbranched and hydrogenated dextrins, useful for the invention have:
from 5% to 40%, preferentially between 10% and 30%, more preferentially between 12% and 22%, and most preferentially between 15 and 20% of 1,6 glucosidic bonds relative to the sum of 1,2, 1,3, 1,4 and 1,6 bonds,
from 42 to 50% of 1,4 glucosidic bonds relative to the sum of 1,2, 1,3, 1,4 and 1,6 bonds,
from 1 to 20% of 1,3 glucosidic bonds relative to the sum of 1,2, 1,3, 1,4 and 1,6 bonds,
from 1 to 20% of 1,2 glucosidic bonds relative to the sum of 1,2, 1,3, 1,4 and 1,6 bonds.

According to one embodiment, the hyperbranched, preferably hyperbranched and hydrogenated dextrins useful for the invention have:
at least 5% of 1,6 glucosidic bonds relative to the sum of 1,2, 1,3, 1,4 and 1,6 bonds, preferentially at least 10%, more preferentially at least 12%, and most preferentially at least 15%,
at most 70% of 1,4 glucosidic bonds relative to the sum of 1,2, 1,3, 1,4 and 1,6 bonds, preferentially at most 60%, and most preferentially at most 50%,
at least 1% of 1,3 glucosidic bonds relative to the sum of 1,2, 1,3, 1,4 and 1,6 bonds, preferentially at least 5%, more preferentially at least 10%, and most preferentially at least 20%,
at least 1% of 1,2 glucosidic bonds relative to the sum of 1,2, 1,3, 1,4 and 1,6, preferentially at least 5%, more preferentially at least 10%, and most preferentially at least 20%.

According to one embodiment, the hyperbranched, preferably hyperbranched and hydrogenated dextrins useful for the invention have:
from 5 to 40%, preferentially from 10 to 30%, more preferentially from 12 to 22%, and most preferentially from 15 to 20% of 1,6 glucosidic bonds relative to the sum of 1,2, 1,3, 1,4 and 1,6 bonds,
from 42 to 70%, preferentially from 42 to 60%, of 1,4 glucosidic bonds relative to the sum of 1,2, 1,3, 1,4 and 1,6 bonds,
from 1 to 20%, preferentially from 1 to 10%, more preferentially from 5 to 15%, and most preferentially from 5 to 10%, of 1,3 glucosidic bonds relative to the sum of 1,2, 1,3, 1,4 and 1,6 bonds,
from 1 to 20%, preferentially from 1 to 10%, more preferentially from 5 to 15%, and most preferentially from 5 to 10%, of 1,2 glucosidic bonds relative to the sum of 1,2, 1,3, 1,4 and 1,6 bonds.

Apart from the use of a well-known pyroconversion process applied to granular starch to prepare dextrins, in other words by using an acid on granular starch, typically at high temperature in a dry environment, these starch branching bond contents may also be obtained by the action of a so-called “branching” or “re-branching” enzyme on a liquefied starch, such as a maltodextrin, a starch hydrolysate or a glucose syrup, optionally previously or subsequently hydrogenated.

These branching bond contents may therefore be obtained both by the action of a so-called “branching” or “re-branching” enzyme on a liquefied starch and/or by the action of an acid typically at high temperature in a dry medium, on a granular or liquefied starch.

Determination of the content of 1,2, 1,3, 1,4 and 1,6 glucosidic bonds may be carried out using the classic methylation technique described in HAKOMORI, S., 1964, J. Biochem., 55, 205. “A rapid permethylation of glycolipid, and polysaccharide catalyzed by methylsulfinyl carbanion in dimethyl sulfoxide”. This method makes it possible to chemically characterize glucosidic bonds by differentiating between free OH groups and bonded groups. This is a destructive method comprising the steps of methylation, hydrolysis, reduction with NaBD4, acetylation and analysis by mass spectrometry.

According to one embodiment, said hyperbranched dextrins have a number-average molecular weight Mn of less than or equal to 10,000 g/mol, preferentially less than or equal to 4500 g/mol, more preferentially between 1000 and 3500 g/mol, even more preferentially between 1500 and 3500 g/mol, and most preferentially between 1800 and 3200 g/mol.

According to one embodiment, said hyperbranched dextrins have a polymolecularity index, the ratio of the weight-average molar mass to the number-average molar mass, noted IP, of less than or equal to 15, preferentially less than or equal to 10, more preferentially less than or equal to 5, and most preferentially less than or equal to 3.

Mn, Mw and IP values are measured by steric exclusion chromatography, based on the size-selective retention of solute molecules due to their penetration or non-penetration into the pores of the stationary phase. The size exclusion chromatography columns used are PSS SUPREMA 100 and PSS SUPREMA 1000, connected in series and coupled to a light scattering detector.

According to one embodiment, said hyperbranched dextrins have a reducing sugar content of less than or equal to 20%, preferentially less than or equal to 15%, more preferentially less than or equal to 10%, even more preferentially less than or equal to 6%, and most preferentially less than or equal to 3% relative to the total mass of the hyperbranched dextrin. The reducing sugar content, expressed as glucose, by weight in relation to the dry weight of the product analyzed, is determined by the BERTRAND method.

According to one embodiment, hyperbranched dextrins useful for the invention have a relatively high content of residual 1,4 glucosidic bonds. This content of 1,4 glucosidic bonds is between 42 and 50% relative to the sum of 1,2, 1,3, 1,4 and 1,6 bonds, in combination with a content of 1,6 glucosidic bonds between 12 and 22% relative to the sum of 1,2, 1,3, 1,4 and 1,6 bonds. In a preferential manner, hyperbranched dextrins useful for the invention have a 1,4:1,6 glucosidic bond ratio of between 1.9 and 4.2 and in particular between 2.3 and 3.5.

According to one embodiment, hyperbranched dextrins useful for the invention have a content of 1,3 glucosidic bonds of between 1 and 10% relative to the sum of 1,2, 1,3, 1,4 and 1,6 bonds, in combination with a content of 1,4 glucosidic bonds of between 42 and 50% relative to the sum of 1,2, 1,3, 1,4 and 1,6 bonds. In a preferential manner, hyperbranched dextrins useful for the invention have a 1,4:1,3 glucosidic bond ratio of between 0.02 and 0.24 and in particular between 0.1 and 0.2. According to one embodiment, hyperbranched dextrins useful for the invention have a 1,2 glucosidic bond content of between 1 and 10%, in combination with a 1,4 glucosidic bond content of between 42 and 50%. In a preferential manner, hyperbranched dextrins useful for the invention have a 1,4:1,2 glucosidic bond ratio of between 0.02 and 0.24 and in particular between 0.1 and 0.2.

According to one embodiment, the hyperbranched dextrins useful for the invention have:
from 5% to 40% of 1,6 glucosidic bonds relative to the sum of 1,2, 1,3, 1,4 and 1,6 bonds,
a reducing sugar content of less than or equal to 20% relative to the total mass of hyperbranched dextrin,
a polymolecularity index less than or equal to 15,
and a number-average molecular weight Mn less than or equal to 4500 g/mol.

According to one embodiment, the hyperbranched dextrins useful for the invention have:
from 10% to 30% of 1,6 glucosidic bonds relative to the sum of 1,2, 1,3, 1,4 and 1,6 bonds,
from 42 to 70% of 1,4 glucosidic bonds relative to the sum of 1,2, 1,3, 1,4 and 1,6 bonds,
a reducing sugar content of less than or equal to 10% of the total mass of hyperbranched dextrin,
a polymolecularity index less than or equal to 10,
and a number-average molecular weight Mn between 1000 and 3500 g/mol.

According to one embodiment, the hyperbranched dextrins useful for the invention have:
from 12% to 22% of 1,6 glucosidic bonds relative to the sum of 1,2, 1,3, 1,4 and 1,6 bonds,
from 42 to 50% of 1,4 glucosidic bonds relative to the sum of 1,2, 1,3, 1,4 and 1,6 bonds,
from 1 to 20% of 1,3 glucosidic bonds relative to the sum of 1,2, 1,3, 1,4 and 1,6 bonds,
from 1 to 20% of 1,2 glucosidic bonds relative to the sum of 1,2, 1,3, 1,4 and 1,6 bonds,
a reducing sugar content of less than or equal to 6% of the total mass of hyperbranched dextrin,
a polymolecularity index less than or equal to 5,
and a number-average molecular weight Mn of between 1500 and 3000 g/mol.

Hyperbranched dextrins useful for the invention are commercially available, such as “Nutriose® FM 06”, “Nutriose® FM 10” and “Nutriose® FM15S” products by Roquette®, or the “Promitor® Soluble fiber” product range by Tate & Lyle.

Other commercial products available under the names of “STA-LITER Polydextrose” from Tate & Lyle, or “Oliggo-fiber®” from Cargill, are hyperbranched polysaccharides or oligosaccharides which, as they stand or after possible hydrogenation, could conceivably exhibit a soothing property like the hyperbranched dextrins according to the object of the present application.

### Hyperbranched and Hydrogenated Dextrins

In a second embodiment, the dextrins useful for the non-therapeutic use forming the object of the present application are hyperbranched and hydrogenated dextrins. Such dextrins may be obtained by subjecting hyperbranched dextrins according to the object of the present application to hydrogenation, or by applying a “branching” or “re-branching” process to a previously hydrogenated dextrin. Hydrogenation may, for example, be achieved by subjecting an aqueous solution of hyperbranched dextrin to hydrogen gas in the presence of a catalyst such as Raney nickel.

An additional feature may thus advantageously be added to the above-mentioned embodiments for the hyperbranched dextrins, namely, a reducing sugar content of less than or equal to 5% by weight, preferentially less than or equal to 3% by weight, more preferentially less than or equal to 2% by weight, more preferentially less than or equal to 1% by weight, more preferentially less than or equal to 0.5% by weight, and most preferentially less than or equal to 0.15% by weight.

According to one embodiment, the hyperbranched and hydrogenated dextrins useful for the invention have:
from 5% to 40% of 1,6 glucosidic bonds relative to the sum of 1,2, 1,3, 1,4 and 1,6 bonds,
a reducing sugar content of less than or equal to 4% relative to the total mass of hyperbranched dextrin,
a polymolecularity index less than or equal to 15,
and a number-average molecular weight Mn less than or equal to 4500 g/mol.

According to one embodiment, the hyperbranched and hydrogenated dextrins useful for the invention have:
from 10% to 30% of 1,6 glucosidic bonds relative to the sum of 1,2, 1,3, 1,4 and 1,6 bonds,
from 42 to 70% of 1,4 glucosidic bonds relative to the sum of 1,2, 1,3, 1,4 and 1,6 bonds,
a reducing sugar content of less than or equal to 3% relative to the total mass of hyperbranched dextrin,
a polymolecularity index less than or equal to 10,
and a number-average molecular weight Mn between 1000 and 3500 g/mol.

According to one embodiment, the hyperbranched and hydrogenated dextrins useful for the invention have:
from 12% to 22% of 1,6 glucosidic bonds relative to the sum of 1,2, 1,3, 1,4 and 1,6 bonds,
from 42 to 50% of 1,4 glucosidic bonds relative to the sum of 1,2, 1,3, 1,4 and 1,6 bonds,
from 1 to 20% of 1,3 glucosidic bonds relative to the sum of 1,2, 1,3, 1,4 and 1,6 bonds,
from 1 to 20% of 1,2 glucosidic bonds relative to the sum of 1,2, 1,3, 1,4 and 1,6 bonds,
a reducing sugar content of less than or equal to 2% relative to the total mass of hyperbranched dextrin,
a polymolecularity index less than or equal to 5,
and a number-average molecular weight Mn of between 1500 and 3000 g/mol.

Hyperbranched and hydrogenated dextrins useful for the invention are commercially available, such as “Nutriose® HM 06” by Roquette.

### Biomarkers Involved in Inflammation (IL-1α, IL-1β, RANTES and VEGF)

Inflammation is essentially an innate self-defense immune response against noxious stimuli, notably infectious agents and physical or chemical challenges. This is a temporary, orchestrated and adapted modification of cellular functions in order to return, if possible, to the state prior to the aggression. The temporal expression profile of cytokines in epidermal keratinocytes is important in orchestrating inflammatory responses (Kataru et al., 2009). IL-1, 6, 8, and TNF-α are known to be potent inducers of keratinocyte-derived VEGF (Detmar et al., 1995).

IL-1 is an essential pro-inflammatory cytokine that mediates the acute phase of inflammation by inducing local and systemic responses. IL-1α functions as an “alarm”. During cell death by necrosis, IL-1α is released into the extracellular space where it stimulates chemokine production, leading to infiltration by neutrophils and then monocytes. IL-1α is constitutively present in the various epithelial cell types of healthy subjects, while IL-1β is mainly induced under pathological conditions.

The mediators RANTES and VEGF are growth factors for connective tissue and vessels.

RANTES has been shown to induce endothelial cell spreading and migration and participate in the formation of vascular networks via VEGF secretion. VEGF is a key mediator of skin angiogenesis and vascular permeability, which is involved in physiological processes such as wound repair and hair growth, as well as in pathological conditions including skin inflammation, skin cancer and psoriasis. In many skin conditions such as psoriasis, contact dermatitis, wound healing and cutaneous neoplasia, which are closely associated with angiogenesis or chronic inflammation, there is a significant induction of VEGF in epidermal keratinocytes, suggesting that increased VEGF plays a key role in these skin problems.

RANTES chemokine is a chemoattractant for eosinophils, memory-phenotype T lymphocytes and monocytes. This chemokine plays an important role in chronic inflammatory and allergic diseases.

Advantageously, it was found that, for these 4 biomarkers involved in the mechanism of inflammation, IL-1α, IL-1β, RANTES AND VEGF, treatment with hyperbranched dextrins induced a decrease in these signals.

These results allow us to conclude that hyperbranched, preferentially hyperbranched and hydrogenated dextrins are effective in preventing or treating at least one symptom selected from redness, heat, swelling and pain, as well as in preventing or treating skin inflammation and mucous membrane inflammation.

### Redness, Heat, Swelling, Pain

The present invention relates to hyperbranched dextrins, preferentially hyperbranched and hydrogenated for use in the prevention or treatment of at least one symptom selected from redness, heat, swelling and pain.

Prevention of at least one symptom selected from redness, heat, swelling and pain means preventing the onset of at least one of these symptoms, as well as delaying the onset of at least one of these symptoms, and preventing the aggravation of at least one of these symptoms.

Treatment of at least one symptom selected from redness, heat, swelling and pain means the disappearance of at least one of the symptoms, or at least a reduction in the intensity of at least one of the symptoms, for example a reduction in the intensity of redness and/or heat and/or swelling and/or pain.

### Epithelial Tissues

According to one embodiment, the at least one symptom is located in at least one epithelial tissue selected from keratinized multi-layered squamous epithelium, non-keratinized multi-layered squamous epithelium, stratified columnar epithelium, ciliated or non-ciliated pseudostratified columnar epithelium. The epidermis is a keratinized multi-layered squamous epithelium.

The oral cavity, vaginal mucosa and urogenital mucosa are non-keratinized, multi-layered squamous epithelia.

The nostrils and conjunctival cul-de-sacs are stratified columnar epithelia.

The nasal mucosa is a ciliated or non-ciliated pseudostratified columnar epithelium.

Thus, according to one embodiment, the at least one symptom is located in the epidermis or the oral cavity or the vaginal mucosa or the nostrils or the conjunctival cul-de-sacs or the nasal mucosa.

According to one embodiment, the at least one symptom is located on the epidermis, preferentially the epidermis of the skin or scalp.

According to one embodiment, the epidermis of the skin or scalp is a sensitive epidermis.

### Skin Inflammation

Skin inflammation is understood to mean the presence of four symptoms on the epidermis, namely, redness, heat, swelling and pain.

The epidermis is preferentially that of the skin or scalp.

According to one embodiment, the epidermis is a sensitive epidermis.

Skin inflammation is also known as dermatitis.

Thus, according to one embodiment, the present invention relates to hyperbranched dextrins, preferentially hyperbranched and hydrogenated dextrins for topical use in the prevention or treatment of symptoms of skin inflammation.

For the purposes of this invention, “topical use” means use on a surface of the body, such as the skin or mucous membranes.

Preventing the symptoms of skin inflammation means not only preventing the onset of the four symptoms of skin inflammation, but also delaying the onset of the four symptoms of skin inflammation, and preventing the four symptoms of skin inflammation from worsening.

Treating the symptoms of skin inflammation means the disappearance of the four symptoms of skin inflammation, or at least a reduction in the intensity of the four symptoms of skin inflammation, for example a reduction in the intensity of redness and heat and swelling and pain.

### Non-Pathological Causes

There are many causes for the appearance of one or all of the symptoms, chosen from redness, heat, swelling and pain.

Thus, the at least one symptom is induced by at least one of the conditions selected from infectious agents, chemicals, ingredients contained in topical compositions for cosmetic, dermocosmetic, dermatological, pharmaceutical, medicinal, veterinary or domestic use (for example detergents), mechanical aggression, environmental factors, thermal aggression, food and beverages. Another non-pathological cause could be an oral or intravenous therapeutic treatment generating one or more secondary skin reactions.

Infectious agents include bacteria, yeasts, molds, parasites, viruses, even mites, lice and insects when they attack the skin through a sting or bite. By way of example, it is possible to cite dry patches caused by parasites.

Mechanical aggressions include rubbing, brushing, shaving, cuts, micro-cuts, physical aggressions, hair removal, abrasion, scrubbing, frequent washing, hard water with high concentrations of lime, etc.

Friction can be caused by clothing rubbing against the skin or by the skin rubbing against the skin. For example, clothing made of wool or tight-fitting garments worn in contact with the skin, such as socks or scarves, rub against the skin, and can thus generate skin irritation. Friction can also be caused by the prolonged wearing of helmets, masks, especially surgical masks, protection against the weather, protection against micro-organisms such as bacteria or viruses, protection against chemicals or solvents or else protection against dust.

Abrasion means bringing skin or mucous membranes into contact with abrasive agents, in other words, solid particles that are generally irregularly shaped and not smooth. For the skin, abrasive agents include gravel, sand and magnesia. For the oral mucosa, abrasive agents may include calcium carbonate.

Brushing refers to the action of a brush, in particular the action of a toothbrush on the gums.

Environmental factors include pollution, exposure to the sun, radiation such as X-rays and UV rays, cold, wind, dryness, humidity, etc.

Thermal aggressions include overexposure to the sun, or exposure to a source of heat or cold.

Foods and beverages include spices, dairy products, dried fruit, nuts, pineapple, acidic foods, vinegar, alcohol, etc.

Chemicals include irritants and pollutants such as detergents, pollution, cigarette smoke, domestic products, and especially household cleaners.

Ingredients contained in topical compositions for cosmetic, dermocosmetic, dermatological, pharmaceutical, medicinal, veterinary or domestic use include cosmetic active ingredients, dermatological active ingredients, surfactants, solvents, preservatives, fragrances, acids, exfoliants and detergents.

These active ingredients with irritant potential have been described in document FR2902998. Examples of dermatological or cosmetic active ingredients thus include certain desquamating agents which can also be peeling agents.

Specific peeling agents include abrasive/exfoliating particles from mineral, organic, natural or synthetic sources. Particularly noteworthy are pumice particles, silica, polyethylene beads, nylon and fruit stone powders.

Among these desquamating agents, the following are likely to cause skin irritation: saturated and unsaturated monocarboxylic acids (acetic acid), saturated and unsaturated dicarboxylic acids, saturated and unsaturated tricarboxylic acids; α-hydroxyacids and β-hydroxyacids of monocarboxylic acids; α-hydroxyacids and β-hydroxyacids of dicarboxylic acids; α-hydroxyacids and β-hydroxyacids of tricarboxylic acids; ketoacids, α-ketoacids and β-ketoacids of polycarboxylic acids, polyhydroxymonocarboxylic acids, polyhydroxybicarboxylic acids and polyhydroxytricarboxylic acids.

Particularly noteworthy among α-hydroxy acids or esters thereof are: glycolic, dioic such as octadecenedioic, citric, lactic, tartaric, malic or mandelic acids, esters thereof such as dialkyl tartrate (C12-C13), branched C12-13 trialcohol citrate.

Among β-hydroxyacids are salicylic acid and derivatives thereof (including n-octanoyl-5-salicylic acid).

The α-keto acids include ascorbic acid and derivatives thereof.

Other desquamating agents include: pyruvic, gluconic, glucuronic, oxalic, malonic, succinic, acetic, gentisic, cinnamic, azelaic acids; phenol; resorcinol; urea and its derivatives, hydroxyethyl urea; oligofucoses; jasmonic acid and derivatives thereof; ascorbic acid and derivatives thereof, trichloroacetic acid; Saphora japonica extract and resveratrol.

From the desquamating agents, those capable of acting on the enzymes involved in desquamation or corneodesmosome degradation may also be likely to cause skin irritation.

These include mineral salt chelating agents such as EDTA; N-acyl-N,N′,N′-ethylenediaminetriacetic acid; aminosulfonic compounds and in particular (N-2 hydroxyethylpiperazine-N-2-ethane) sulfonic acid (HEPES); 2-oxothiazolidine-4-carboxylic acid derivatives (procysteine); glycine-type alpha-amino acid derivatives; honey; sugar derivatives such as O-octanoyl-6-D-maltose, O-linoleyl-6-D-glucose and N-acetyl glucosamine.

Retinoids are also compounds that can cause skin irritation. Examples include retinol and esters thereof, retinal, 30 retinoic acid and derivatives thereof, and adapalene.

Salts and derivatives, such as cis- or trans-forms, racemic mixtures, dextrorotatory or levorotatory forms of the above-mentioned compounds are also considered compounds likely to cause skin irritation.

Other dermatological or cosmetic active ingredients likely to cause skin irritation are also listed below:
urea and derivatives thereof such as hydroxyethyl urea,
certain vitamins such as vitamin D and derivatives thereof such as vitamin 10 D3, vitamin D2, calcitriol, calcipotriol, tacalcitol, 24,25-diOH vitamin D3, 1-OH vitamin D2 and 1,24-diOH vitamin D2; vitamin B9 and derivatives thereof,
peroxides such as benzoyl peroxide and hydrogen peroxide,
hair loss inhibitors such as minoxidil and derivatives thereof such as aminexyl,
hair dyes and colorants such as aminophenols and derivatives thereof such as para-phenylenediamine (p-PDA), N-phenyl p-PDA, toluene 2,5-diamine sulfate, meta-phenylenediamine (m-PDA), toluene 3,4-diamine and ortho-phenylenediamine (o-PDA),
antiperspirants like aluminum salts, such as aluminum hydroxychloride,
deodorants,
   depilatory and/or perming active ingredients such as thioglycolates and ammonia,
thioglycolate and salts thereof,-phenoxyethanol,
1,2-pentanediol,
alcoholic fragrance solutions (perfumes, eau de toilette, aftershaves, deodorants)
anthralins (dioxyanthranol),
anthranodes
lithium salts,
depigmenting agents (e.g.: hydroquinone, high-concentration Vitamin C, kojic acid),
certain slimming active ingredients with a heating effect,
nicotinates and derivatives thereof,
capsaicin, anti-lice active ingredients (pyrethrin),
antiproliferatives such as 5-fluorouracil or methotrexate,
antiviral agents,
antiparasitics,
antifungals,
antipruritic agents,
anti-seborrheic agents,
certain sunscreens,
propigmenting agents such as psoralens and methylangecilines, and
mixtures thereof.

Preservatives include propionic acid, calcium propionate, formaldehyde, paraformaldehyde, o-phenylphenol or salts thereof, zinc pyrithione, sodium sulfites, bisulfites or metabisulfites, ammonium, potassium, chlorobutanol, methylparaben, ethylparaben, propylparaben, formic acid and salts thereof such as sodium formate, benzoic acid and salts thereof such as sodium benzoate, sorbic acid and salts thereof such as calcium sorbate, sodium sorbate, potassium sorbate, salicylic acid and salts thereof, dehydroacetic acid and salts thereof such as sodium dehydroacetate, undecylenic acid and salts thereof, such as calcium undecylenate, potassium undecylenate, sodium undecylenate, phenoxyethanol, 1,2-Dimethylol-5,6-dimethylhydantoin, benzyl alcohol, chlorhexidine, chlorhexidine diacetate, chlorhexidine digluconate, chlorhexidine dihydrochloride, behentrimonium chloride, cetrimonium chloride, cetrimonium bromide, laurtrimonium chloride, laurtrimonium bromide, steartrimonium chloride, steartrimonium bromide, hexamidine, hexamidine diisethionate, chlorphenesin, benzalkonium chloride, benzalkonium bromide, benzalkonium saccharinate, ethyl lauroyl arginate.

Surfactants include anionic, cationic and amphoteric surfactants, more particularly anionic surfactants such as alkyl sulfates and alkyl ether sulfates like lauryl sulfate and lauryl ether sulfate, and salts thereof, especially sodium salts.

In a preferred embodiment of the invention, the compound likely to cause skin irritation is selected from surfactants, bases and acids. The acids may be selected from citric acid, lactic acid, acetic acid, dehydroacetic acid, formic acid, gluconic acid, succinic acid, salicylic acid, glucuronic acid, retinoic acid, glycolic acid, phytic acid, ascorbic acid and levulinic acid. Bases can be selected from sodium hydroxide, potassium hydroxide, triethanolamine and aminomethyl propanol.

Preferentially, the at least one symptom selected from redness, heat, swelling and pain is induced by chemicals, ingredients contained in cosmetic or dermatological formulations, mechanical aggression, thermal aggression or environmental factors.

More preferentially, the at least one symptom selected from redness, heat, swelling and skin pain or inflammation is induced by exposure to chemicals or ingredients contained in topical compositions for cosmetic or dermatological use selected from surfactants, detergents, hair oxidation or bleaching agents, or is induced by mechanical aggression selected from rubbing, shaving, cuts, micro-cuts, hair removal, abrasion and exfoliation.

There are also numerous non-pathological causes of skin inflammation. In fact, skin inflammation may be induced by at least one of the causes previously described and selected from infectious agents, chemical products, ingredients contained in topical compositions for cosmetic, dermocosmetic, dermatological, pharmaceutical, medicinal, veterinary or domestic use, mechanical aggressions, environmental factors, thermal aggressions, food and beverages, but also by skin irritation, skin corrosion, endogenous skin inflammation, or a combination of these causes.

Skin irritation Skin irritation includes acute skin irritation (or acute irritant contact dermatitis), cumulative skin irritation (or cumulative irritant contact dermatitis) and phototoxicity (or phototoxic dermatitis).

Acute skin irritation” or “acute irritant contact dermatitis” is understood to mean a reversible local inflammatory response of the skin of a normal living being to a direct injury caused by the single application of a toxic substance, without the intervention of an immunological mechanism.

“Cumulative skin irritation” or “cumulative irritant contact dermatitis” is understood to mean reversible irritation resulting from repeated or continuous exposure to materials that do not in themselves cause acute irritation.

“Phototoxicity” or “phototoxic dermatitis” is understood to mean irritation resulting from light-induced molecular changes in the structure of chemicals applied to the skin.

Thus, according to one embodiment, skin irritation is selected from cumulative irritant contact dermatitis, acute irritant contact dermatitis and phototoxic dermatitis.

In a preferred embodiment, the skin irritation is cumulative irritant contact dermatitis.

“Skin corrosion” is understood to mean direct chemical action on normal living skin leading to its disintegration and irreversible alteration at the point of contact. Corrosion manifests itself as ulceration and necrosis with subsequent formation of scars.

According to one embodiment, phototoxicity is induced by chemicals, ingredients contained in topical compositions for cosmetic or dermatological use.

In a preferred embodiment, skin inflammation is induced by exposure to chemicals and ingredients contained in topical compositions for cosmetic or dermatological use, mechanical aggression and thermal aggression.

In a preferred embodiment, skin inflammation is induced by exposure to chemicals and ingredients contained in topical compositions for cosmetic or dermatological use and preferentially by ingredients contained in topical compositions for cosmetic or dermatological use.

“Endogenous skin inflammation” is understood to mean inflammation induced by one or more biological factors internal to the human organism, or by one or more pathogenic agents endogenous to the human organism, in other words, pathogenic agents synthesized by the human organism itself.

### Causes of Mucosal Inflammation

According to one embodiment, the invention relates to hyperbranched dextrins, preferentially hyperbranched hydrogenated dextrins for use in the prevention or treatment of the symptoms of mucosal inflammation.

According to one embodiment, inflammation is selected from inflammation of the oral mucosa, preferentially inflammation of the oral mucosa by anionic surfactants, or inflammation of the oral mucosa due to irritation caused by tooth brushing. Said anionic surfactants may be applied to the oral mucosa by means of a toothpaste, generally with concomitant brushing, or a mouthwash, generally without concomitant brushing.

Thus, according to one embodiment, inflammation of the mucous membranes is induced by ingredients contained in topical compositions for cosmetic, dermocosmetic, dermatological, pharmaceutical, medicinal, veterinary or domestic use, such as surfactants or by mechanical aggression such as brushing.

### Pathological Causes

According to one embodiment, skin inflammation is induced by a dermatological pathology involving an inflammatory process.

According to one embodiment, the dermatological pathology involving an inflammatory process is selected from psoriasis, cutaneous atopy, atopic dermatitis, immediate hypersensitivity type allergic reactions, delayed hypersensitivity type allergic reactions, inflammatory hyperpigmentation, immune dermatoses, actinic elastosis, alopecia areata, vitiligo, systemic lupus erythematosus, pemphigus vulgaris, dystrophic epidermolysis and autoimmune canities, erythema,

Immediate hypersensitivity type allergic skin reactions include urticaria, lucitis (or sun allergy) and allergies due to exposure to surfactants.

Delayed hypersensitivity type allergic skin reactions include contact dermatitis, seborrheic dermatitis, acne and eczema.

Erythema can be caused by ultraviolet rays, rubbing or micro-cuts.

According to one embodiment, atopic dermatitis is atopic dermatitis of sensitive skin.

Preferentially, dermatological pathologies are selected from erythema, psoriasis, cutaneous atopy, atopic dermatitis, immediate hypersensitivity type allergic reactions, delayed hypersensitivity type allergic reactions and inflammatory hyperpigmentation.

Preferentially, erythema is caused by ultraviolet light.

Preferentially, immediate hypersensitivity type allergic reaction is urticaria.

Preferentially, delayed hypersensitivity type allergic reactions are selected from contact dermatitis and eczema.

According to one embodiment, mucosal inflammation is induced by a dermatological pathology involving an inflammatory process.

According to one embodiment, dermatological pathologies are selected from pathologies of the oral mucosa, vaginal mucosa and urogenital mucosa, preferentially from pathologies of the oral mucosa.

According to one embodiment, oral mucosal pathologies are selected from gingivitis, oral lichen planus, mouth ulcers, etc.

The terms “prevention” or “treatment” or “prevention method” or “treatment method” are not absolute terms but refer to a procedure or plan of action designed, even with a low probability of success, to induce an overall beneficial effect such as the disappearance of the pathology, but also the delay in the onset of symptoms of the pathology, or the reduction in the severity of one or more symptoms. Typically, in the case of skin inflammation induced by a dermatological pathology involving an inflammatory process, prevention means not only preventing the symptoms of the pathology but also preventing the symptoms of the pathology from worsening.

The present invention also relates to a method for the prevention or treatment of the symptoms of skin inflammation induced by a dermatological pathology involving an inflammatory process comprising the administration of hyperbranched dextrins, preferentially hyperbranched and hydrogenated dextrins, to a patient in need thereof.

The present invention also relates to the use of hyperbranched dextrins, preferentially hyperbranched and hydrogenated dextrins, to obtain a medicine for the prevention or treatment of the symptoms of skin inflammation induced by a dermatological pathology involving an inflammatory process.

The present invention also relates to a composition comprising hyperbranched dextrins, preferentially hyperbranched and hydrogenated dextrins, for the therapeutic use thereof in the prevention or treatment of the symptoms of skin inflammation induced by a dermatological pathology involving an inflammatory process.

Prevention and/or Reduction of the Irritant Effect of Chemicals or Ingredients Contained in Topical Compositions for Cosmetic, Dermocosmetic, Dermatological, Pharmaceutical, Medicinal, Veterinary or Domestic Use

According to one embodiment, the invention relates to the use of hyperbranched dextrins, preferentially hyperbranched and hydrogenated dextrins, to prevent, reduce or eliminate the irritant effect of chemical products coming into contact with the skin and mucous membranes, or of ingredients applied topically, contained in topical compositions for cosmetic, dermocosmetic, dermatological, pharmaceutical, medicinal, veterinary or domestic use.

An irritant effect means the appearance of at least one symptom in epithelial tissue or mucous membranes, selected from redness, heat, swelling and pain following exposure of the skin or mucous membrane to one or more chemicals, or following topical application of cosmetic, dermocosmetic, dermatological, pharmaceutical, medicinal, veterinary or domestic compositions comprising ingredients that irritate epithelial tissue or mucous membranes.

Hyperbranched dextrins, preferentially hyperbranched and hydrogenated dextrins, can be applied before or after the use of these ingredients or active ingredients with an irritant effect, in order to prevent, greatly attenuate, or even eliminate this irritant effect. In both cases, the hyperbranched dextrin, preferentially hyperbranched and hydrogenated, will be in a topical formulation for cosmetic, dermocosmetic or dermatological, pharmaceutical, medicinal or veterinary use, which, in fact, will not contain any ingredient or active ingredient with an irritant effect.

The presence of hyperbranched dextrins, preferentially hyperbranched and hydrogenated dextrins, in a topical composition for cosmetic, dermocosmetic, dermatological, pharmaceutical, medicinal, veterinary or domestic use, preferentially in a topical composition for cosmetic, dermocosmetic or dermatological use, comprising one or more ingredients or active ingredients with an irritant effect also makes it possible to greatly attenuate, or even eliminate this irritant effect. This also makes it possible to increase the amount of product with an irritant side effect compared with the amount of product normally used, for improved efficacy. This also makes it possible to administer products with an irritating effect to sensitive skin.

Topical compositions for cosmetic, dermocosmetic, dermatological, pharmaceutical, medicinal, veterinary or domestic use may contain one or more ingredients or active ingredients likely to cause skin irritation, such as cosmetic or dermatological active ingredients, surfactants, solvents, preservatives, perfumes and detergents.

These ingredients or active ingredients with irritant potential are as previously described.

### Dermatological Composition or Dermatologically Acceptable Composition

Hyperbranched dextrins, preferentially hyperbranched and hydrogenated, may be incorporated into a dermatological composition, or more generally into any composition made from a dermatologically acceptable medium and brought into contact with an epithelial tissue selected from keratinized multi-layered squamous epithelium, non-keratinized multi-layered squamous epithelium, stratified columnar epithelium, ciliated or non-ciliated pseudostratified columnar epithelium, preferentially, preferentially being brought into contact with the epidermis, oral cavity, oral mucosa, vaginal mucosa, nostrils, nasal mucosa, and most preferentially being brought into contact with the skin or oral mucosa.

Thus, the present invention also relates to a dermatological or dermatologically acceptable composition comprising hydrogenated and hyperbranched dextrins and a dermatologically acceptable medium.

The term “dermatologically acceptable composition” or “dermatologically acceptable” is understood to mean any composition that does not or very rarely has deleterious side effects and in particular does not produce unacceptable redness, heat, tightness or tingling, or more generally inflammation, for a user who brings said dermatologically acceptable composition into contact with epithelial tissue, preferentially with the skin or mucous membranes of said user. The dermatologically acceptable composition is thus compatible with human epithelial tissue, and mucous membranes of humans. Dermatologically acceptable compositions include pharmaceutical, medicinal and domestic compositions, including detergent compositions.

The composition according to the invention may also be another topical composition intended to treat, preventively or curatively, at least one epithelial tissue selected from keratinized multi-layered squamous epithelium (epidermis), non-keratinized multi-layered squamous epithelium (oral cavity, vaginal mucosa), stratified columnar epithelium (nostrils, conjunctival cul-de-sac), ciliated or non-ciliated pseudostratified columnar epithelium (nasal mucosa). Consequently, this composition may be a topical composition for dermatological, pharmaceutical, medicinal or veterinary use, for skin and epithelial tissue care.

### Forms of Administration

The dermatological composition according to the invention may be in any pharmaceutical form normally used for topical application such as aqueous, hydroalcoholic or oily solutions, lotion- or serum-type dispersions or solutions, milk-type emulsions of liquid or semi-liquid consistency, obtained by dispersion of a fatty phase in an aqueous phase (O/W) or vice-versa (W/O), or of soft, semi-solid or solid consistency, cream-type emulsions or suspensions, aqueous or anhydrous gels, anhydrous compositions, solid compositions of microemulsions, microcapsules, microparticles, or ionic and/or nonionic vesicular dispersions.

These compositions are prepared according to the usual methods known to the person skilled in the art.

The composition may be applied before, during or after the onset of at least one symptom selected from redness, heat, swelling and pain, or inflammation of the skin or mucous membranes, and said application may be repeated or renewed as required.

This composition, when applied to the skin, may be more or less fluid and have the appearance of a cream, an emulsion or microemulsion, an ointment, a milk, a lotion, a serum, a paste, a foam, a mask, a fluid, a balm, an oil, a gel or a salve. It may optionally be applied to the skin in aerosol form. It may also be presented in solid form, and for example in stick or patch form, dry soaps or cleansing bars.

It may be used as a skin care product, particularly for leave-on application, such as a day or night cream for facial and/or body skin, as a cleanser, as a make-up product, as a deodorant, as a shaving product, as an aftershave product, as an after-sun product, as a sun protection product, or as a lip balm to protect lips from the cold and/or the sun and/or the wind.

The composition according to the invention may also be a composition for scalp and hair care and may be in the pharmaceutical form for rinse-off or non-rinse-off application. Examples of these forms are shampoos, hair conditioners, hair masks, serums, mousses, balms, creams, sprays, conditioners, detanglers, hair creams and also in the form of treatment lotions, dye compositions, coloring shampoos, perm compositions, anti-hair loss lotions or gels, anti-parasitic shampoos, or conditioners.

The amount of hyperbranched dextrins in the compositions may be varied to deliver an effective amount of hyperbranched dextrins to achieve the desired therapeutic response for a particular patient.

“Effective amount” or “therapeutically effective amount” of a compound is understood to mean a non-toxic yet sufficient quantity of the compound to provide the desired effect.

Typically, the amount administered or dose depends on the activity of the hyperbranched dextrins, the route of administration, the severity of the pathology, as well as the health status and medical history of the patient being treated, various factors such as body weight, diet, and possible combination with other therapeutic agents. However, it is within the competence of the skilled person to determine the appropriate dosage and to initiate treatment at a dosage lower than that required to obtain the desired therapeutic effect and to gradually increase the dose until the desired effect is obtained.

According to one embodiment, the content of hyperbranched dextrins ranges from 0.5 to 50% by weight, preferentially from 1 to 25% by weight, more preferentially from 1 to 15% by weight, even more preferentially from 2 to 10% by weight, based on the total weight of the cosmetic, dermatological or dermatologically acceptable composition.

### Formulation Ingredients

According to one embodiment, hyperbranched dextrins are administered topically.

Hyperbranched dextrins may be administered in several doses.

The dermatological composition or topical formulation according to the invention may also comprise a solvent selected according to the different ingredients and the form of administration.

According to one embodiment, the composition according to the invention may comprise an aqueous phase comprising water and optionally, one or more water-miscible organic solvents.

The term “water-soluble solvent” refers to a compound that is liquid at room temperature and miscible with water (miscibility in water greater than 50% by weight at 25° C. and atmospheric pressure).

Water-soluble solvents that can be used in the compositions according to the invention can be volatile.

Among the water-soluble solvents that can be used in the compositions in accordance with the invention, mention may be made of monoalcohols having from 1 to 5 carbon atoms such as ethanol and isopropanol, C3-C4 ketones and C2-C4 aldehydes and polyols such as glycerol or glycerin, sorbitol or isosorbide.

The cosmetic composition may also comprise, in addition to hydrogenated and highly branched dextrins:

### —One or More Humectants:

The humectant agent(s) will be selected from polyols and/or esters of fatty acids and of polyethylene glycol.

“Polyols” is understood to mean any molecule having in its structure at least two free hydroxy (—OH) groups. These polyols are preferably liquid at room temperature (25° C.).

Typically, the polyol will be selected from maltitol, mannitol, xylitol, erythritol, sorbitol, isosorbide, glycerol or glycerin, glucose, sucrose, polydextrose, hydrogenated glucose syrups, dextrins, maltodextrins, glucose syrups, and mixtures thereof.

Examples include Beauté by Roquette® PO 071 (INCI: Sorbitol), Beauté by Roquette® PO 260 (INCI: Mannitol), Beauté by Roquette® PO 370 (INCI: Xylitol), Beauté by Roquette® PO 455 (INCI: Hydrogenated starch hydrolysate), Beauté by Roquette® PO 500 (INCI: Isosorbide), all sold by ROQUETTE, glycerin (INCI: glycerin) sold by COOPER, propylene glycol (INCI: propylene Glycol) sold by COOPER., Butylene Glycol (INCI: 1,3-BUTANEDIOL)

Among the esters of fatty acids and of polyethylene glycol, one may mention the product sold under the name Glucamate SSE-20 (INCI: PEG-20 METHYL GLUCOSE SESQUISTEARATE) by the company LUBRIZOL ADVANCED MATERIALS, Inc.

The composition comprises from 0.5% to 25% by weight of one or more humectants, preferably, from 1% to 15% by weight, and even more preferably, from 2% to 10% by weight of polyols, relative to the total weight of the composition.

### —One or More Oils

For the purposes of the present invention, “oil” is understood to mean a compound which is liquid at room temperature (25° C.) and which, when it is introduced at an amount of at least 1% by weight into the water at 25° C., is not at all soluble in the water, or soluble to an extent of less than 10% by weight, relative to the weight of oil introduced into the water.

According to one embodiment, the oil will be chosen from volatile oils, non-volatile oils and mixtures thereof. Preferably, these oils are vegetable or of vegetable origin.

“Non-volatile oil” is understood to mean an oil remaining on the keratin materials at room temperature and atmospheric pressure for at least several hours and especially having a vapor pressure of less than 103 mmHg (0.13 Pa).

Among non-volatile oils, mention may be made of fatty esters such as cetearyl isononoate, isotridecyl isononoate, isostearyl isostearate, isopropyl isostearate, isopropyl myristate, isopropyl palmitate, butyl stearate, hexyl laurate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, triacetin, tricaprin, caprylic/capric acid triglycerides, coco caprate and caprylate mixture, C12 to C15 alkyl benzoates, glycol esters such as butylene glycol cocoate, glyceryl triisostearate, tocopherol acetate, higher fatty acids such as myristic acid, palmitic acid, stearic acid, behenic acid, oleic acid, linoleic acid, linolenic acid or isostearic acid, higher fatty alcohols such as oleic alcohol, vegetable oils such as avocado oil, camellia oil, hazelnut oil, tsubaki oil, cashew nut oil, argan oil, soybean oil, grape seed oil, sesame oil, corn oil, wheatgerm oil, rapeseed oil, sunflower oil, cottonseed oil, jojoba oil, peanut oil, macadamia oil, sweet almond oil, olive oil and mixtures thereof.

These non-volatile oils may also be hydrocarbon or silicone type oils, such as paraffin oils, squalane, petroleum jelly, dimethyl siloxanes and mixtures thereof.

According to one embodiment, the non-volatile oil is selected from stearic acid, jojoba oil (INCI: Simmondsia Chinensis Seed Oil), grape seed oil (INCI: Vitis vinifera seed oil), macadamia oil (INCI: Macadamia ternifolia seed oil), refined oleic sunflower oil (Helianthus annuus seed oil), the mixture of caprate and coco caprylate such as the product Miglyol Coco 810 (INCI: Coco-Caprylate/Caprate), sweet almond oil (INCI: Prunus Amygdalus Dulcis Oil), sesame oil (INCI: Sesamum indicum seed oil).

Volatile oil is intended to mean an oil which is capable of evaporating from the skin in less than one hour at room temperature and atmospheric pressure. The volatile oils may for example be selected from silicone oils or short-chain fatty acid triglycerides in order to reduce the greasy feel.

According to one embodiment, the oil(s) is/are present at a content ranging from 0.5% to 70% by weight, preferably from 1% to 60% by weight, preferably from 2% to 50% by weight, preferably 5% to 40% by weight, preferably 5% to 30% by weight relative to the total weight of the composition.

—One or More Waxes and/or One or More Pasty Compounds.

“Wax” is intended to mean a fatty substance having reversible liquid-solid state change and having a melting point greater than 25° C., generally between 30° C. and 90° C., which is liquid under the preparation conditions of the composition and which has anisotropic crystalline organization in the solid state. The waxes used according to the invention may consist of polar or apolar waxes or a mixture of these two. “Apolar” is understood to mean a wax containing only carbon, hydrogen and/or phosphorus atoms and in particular a hydrocarbon.

The polar waxes may be selected from animal waxes, vegetable waxes, and synthetic or silicone waxes containing polar groups such as esters. Mention may thus be made of carnauba wax, candelilla wax, beeswax (Cera alba), Chinese insect wax (Ericerus pela), Japan wax, sumac wax, montan wax, triesters of C8-C20 acids and glycerin, such as glyceryl tribehenate, acetylated glycol stearate, sold particularly by VEVY under the trade name CETACENE, and mixtures thereof. These waxes may in particular be used in predispersed form in an oil, as is the case of the mixture of candelilla wax and jojoba seed oil. Preferably, these waxes are vegetable or of vegetable origin.

According to one embodiment, the wax or waxes is/are present at a content ranging from 0.5 to 50% by weight, preferably from 1 to 25% by weight, preferably from 5 to 10% by weight, relative to the total weight of the composition.

“Pasty compound” is understood to mean lipophilic fatty substances which, like waxes, are capable of undergoing a reversible liquid-solid state change and which have, in the solid state, anisotropic crystalline organization, but which differ from waxes in that they contain, at a temperature of 23° C., a liquid fraction and a solid fraction. These pasty compounds are preferably vegetable butters or butters of vegetable origin, such as shea butter or camellia butter.

According to one embodiment, the pasty compound(s) is/are present at a content ranging from 0.5 to 50% by weight, preferably from 1 to 25% by weight, preferably from 5 to 10% by weight, relative to the total weight of the composition.

### —One or More Gelling Agents

A gelling agent is understood to mean a compound which, in the presence of a solvent, creates more or less strong intermacromolecular bonds, thus inducing a three-dimensional network which fixes said solvent. Gelling agent is also understood to mean thickening or rheological agents that act on the viscosity and flow properties of an aqueous phase or a fatty phase.

The gelling agent particularly allows the viscosity of the continuous phase to be increased, for example to adjust the viscosity of the continuous phase to a value ranging from 100 mPa·s to 20,000 mPa·s.

The gelling agent may be selected from polymers of synthetic origin or of plant origin, preferentially of plant origin, chemically modified or not. It may thus be selected from gums derived from plants such as gum arabic, konjac gum, guar gum or derivatives thereof, gums extracted from algae such as alginates; gums derived from microbial fermentation such as xanthans, for example, the product Keltrol CG (INCI: xanthan gum), sold by the company CP KELCO or the product Xanthan Gum FNCS-PC (INCI: xanthan gum) sold by the company JUNGBUNZLAUER INTERNATIONALAG, mannans, scleroglucan or derivatives thereof; cellulose and derivatives thereof such as carboxymethylcellulose or hydroxyethylcellulose; starch and derivatives thereof such as modified starches, notably pregelatinized, acetylated, hydroxypropylated, carboxymethylated, cationic, octenylsuccinates, branched starches, optionally modified several times; synthetic polymers such as polyacrylic acids or carbomers, and mixtures thereof.

Among the starches and starch-based blends, mention should be made of Beauté by Roquette® ST 118 (INCI: Carboxymethyl starch), Beauté by Roquette® ST 720 (INCI: Hydroxypropyl starch) and Beauté by Roquette® DS112 (INCI: Starch acetate (and) Hydroxyethylcellulose (and) Xanthan gum), all sold by Roquette.

Among the carbomers, mention will for example be made of the product Carbopol Ultrez 30 (INCI: carbomer) from the company LUBRIZOL ADVANCED MATERIALS, Inc. or CARBOPOL ETD 2050 POLYMER (INCI: carbomer).

Mention will also be made of mixtures of gum arabic and xanthan gum such as the product Solagum AX (INCI: Acacia Senegal gum (and) Xanthan gum), sold by the company SEPPIC, mixtures of starch and cellulose derivatives such as the product Beauté by ROQUETTE® DS112 (INCI: Starch acetate (and) Hydroxyethylcellulose (and) Xanthan gum), sold by the company ROQUETTE.

The gelling agent may be a mineral gelling agent selected from magnesium and/or aluminum silicates. An example of such a mineral gelling agent is Veegum® Pure from Vanderbilt Minerals LLC, which is a magnesium aluminum silicate.

According to one embodiment, the gelling agent(s) is/are present at a content ranging from 0.1 to 50% by weight, preferably from 0.5 to 25% by weight, preferably from 0.3 to 15% by weight, relative to the total weight of the composition.

### —One or More Binders

The term “binder” means compounds conferring increased cohesion of the cosmetic composition. This cohesion can be adjusted as a function of the amount and the chemical affinity of the binder relative to the ingredients of the cosmetic composition.

Typically, the binder will be chosen from caprylic/capric acid triglicerides such as the product Labrafac CC (INCI: capric/caprylic triglicerides) or Cetyl Dimethicone (INCI).

The binder(s) is/are present in the composition according to the invention at a content ranging from 0.5% to 20%, preferably from 1 to 15%, preferably from 5 to 12%, by weight relative to the total weight of the composition.

### —One or More Emulsifying Agents

The cosmetic composition according to the invention may also comprise one or more oil-in-water (O/W) or water-in-oil (W/O) emulsifiers.

The oil-in-water (O/W) emulsifier is an emulsifying agent with a HLB greater than or equal to 8 and be selected from sorbitan esters which could be polyethoxylated, glycerol fatty acid esters, sucrose fatty acid esters or polyesters, polyethylene glycol fatty acid esters, polyether-modified polysiloxanes, polyethyleneglycol fatty alcohol ethers, alkyl polyglycosides and hydrogenated lecithin, fatty acids, sorbitan esters, this list being non-limiting and mixtures thereof.

Examples include Montanov 68 (INCI: CETEARYL ALCOHOL (AND) CETEARYL GLUCOSIDE), sold by SEPPIC, Montanov L (INCI: C14-C22 Alcohols & C12-20 Alkyl Glucoside), sold by SEPPIC, Montanov 202 (INCI: Arachidyl Alcohol (and) Behenyl Alcohol (and) Arachidyl Glucoside), Citrol GMS 40 (INCI: Glyceryl stearate), sold by CRODA, or Imwitor 960K (INCI: Glyceryl stearate), sold by BIESTERFELD, the product Imwitor 372P (INCI: Glyceryl stearate citrate), sold by BIESTERFELD, the product Glucate SS (INCI: Methyl Glucose Sesquistearate), sold by LUBRIZOL ADVANCED MATERIALS, Inc, plural stearic (Polyglyceryl-6 Distearate), Natragem E145 (INCI: Polyglyceryl-4 Laurate/Succinate (and) Aqua), sold by CRODA, Span 60 (INCI: sorbitan stearate), sold by SIGMA ALDRICH.

Among the sorbitan esters, mention will for example be made of the product Span 20.

Among the fatty alcohols, mention will for example be made of the product sold under the name Promulgen D (INCI: Cetearyl alcohol (and) Ceteareth-20).

The O/W emulsifier may advantageously be selected from emulsifier systems composed of a cyclodextrin and a water-in-oil emulsifier of natural origin, such as the emulsifier system marketed by Roquette Frères under the name Beauté by Roquette® DS 146, or from emulsifier systems based on modified starches and plant-based gums, such as Beauté by Roquette® DS 421 sold by the applicant.

The water-in-oil (W/O) emulsifier is an emulsifier agent with a HLB less than 8 and selected from non-ethoxylated polyol fatty esters, and especially from non-ethoxylated fatty esters of glycerol, polyglycerols, sorbitol, sorbitan, anhydrohexitols, such as in particular isosorbide, mannitol, xylitol, erythritol, maltitol, sucrose, glucose, polydextrose, hydrogenated glucose syrups, dextrins and hydrolyzed starches.

The emulsifying agent(s) is/are present in the composition according to the invention at a content ranging from 0.5% to 20%, preferably from 1 to 15%, preferably from 2 to 12%, by weight relative to the total weight of the composition.

### One or More Surfactants

According to one embodiment, the composition according to the invention comprises at least one surfactant selected from ionic, non-ionic, anionic, cationic or amphoteric or zwitterionic surfactants. These surfactants are selected for their detergent and foaming function.

In a further embodiment, the composition according to the invention comprises at least one surfactant selected from anionic surfactants, amphoteric surfactants or zwitterionic surfactants.

The anionic surfactants are chosen from carboxylic anionic surfactants, sulfate-based anionic surfactants, sulfonate surfactants, phosphate anionic surfactants, and mixtures thereof, preferably from sulfonate anionic surfactants, carboxylic anionic surfactants, and mixtures thereof.

“Anionic surfactant” is intended to mean a surfactant which only comprises anionic groups as ionic or ionizable groups. In the present description, an entity is described as being “anionic” when it has at least one permanent negative charge or when it can be ionized into a negatively charged entity, under the conditions of use of the composition of the invention (medium, pH for example) and not comprising any cationic charge. “Sulfate-based anionic surfactant” is intended to mean an anionic surfactant comprising at least one sulfate function (—OSO₃H or —OSO₃⁻), and may optionally further comprise one or more other functions derived from acids, such as carboxylic acid or carboxylate functions (—COOH or —COO⁻), sulfonate functions (—SO₃H or —SO₃⁻) and/or phosphate functions. By way of example, alkyl sulfates, alkyl ether sulfates, alkylamidoether sulfates, alkylarylpolyethersulfates, monoglyceride sulfates, and also the salts of these compounds, are sulfate-based anionic surfactants. The alkyl groups of these compounds cited by way of example comprise 6 to 30 carbon atoms, and the aryl group denotes a phenyl or benzyl group. These compounds cited by way of example may be polyoxyalkylenated, in particular polyoxyethylenated, and comprise from 1 to 50 ethylene oxide units. “Non-sulfate-based anionic surfactant” is intended to mean a surfactant which does not fall within the definition of “sulfate-based anionic surfactant” as defined above.

It is also intended, according to the invention, that:
the carboxylic anionic surfactants comprise at least one carboxylic or carboxylate function (—COOH or —COO⁻), but does not comprise a sulfonic or sulfonate function (—SO₃H or —SO₃⁻), nor sulfate function (—OSO₃H or —OSO₃⁻);
the sulfonate anionic surfactants comprise at least one sulfonic or sulfonate function (—SO₃H or —SO₃⁻), and may optionally further comprise one or more carboxylic or carboxylate functions (—COOH or —COO⁻) and/or phosphates, but do not comprise a sulfate function (—OSO₃H or —OSO₃⁻).
the phosphate anionic surfactants comprise at least one phosphoric or phosphate function (—OPO₃H₂ or —OPO₃²⁻), but do not comprise a carboxylic or carboxylate function (—COOH or —COO⁻), nor a sulfonic or sulfonate function (—SO₃H or —SO₃⁻), nor a sulfate function (—OSO₃H or —OSO₃⁻).

In other words:
an anionic surfactant comprising at least one sulfate function (—OSO₃H or —OsO₃⁻), and at least one carboxylic or carboxylate function (—COOH or —COO⁻), is considered, within the meaning of the invention and unless otherwise indicated, as a sulfate-based anionic surfactant;
an anionic surfactant comprising at least one sulfate function (—OSO₃H or —OSO₃⁻), and at least one sulfonic or sulfonate function (—SO₃H or —SO₃⁻), is considered, within the meaning of the invention and unless otherwise indicated, as a sulfate-based anionic surfactant;
an anionic surfactant comprising at least one sulfonic or sulfonate function (—SO₃H or —SO₃⁻), and at least one carboxylic or carboxylate function (—COOH or —COO⁻), is considered, within the meaning of the invention and unless otherwise indicated, as a non-sulfated sulfonate anionic surfactant; • an anionic surfactant comprising at least one sulfate function (—OSO₃H or —OSO₃⁻), and at least one sulfonic or sulfonate function (—SO₃H or —SO₃⁻), and at least one carboxylic or carboxylate function (—COOH or —COO⁻), is considered, within the meaning of the invention and unless otherwise indicated, as a sulfate-based anionic surfactant.

The carboxylic anionic surfactants can be chosen from the following compounds: acylglycinates, acyllactylates, acylsarcosinates, acylglutamates, alkyl-D-galactoside-uronic acids, alkyl ether carboxylic acids, alkyl(aryl)ether carboxylic acids, alkyl(amido) ether carboxylic acids; and the salts of these compounds. The alkyl and/or acyl groups of these compounds comprise from 6 to 30 carbon atoms, more preferentially from 8 to 28, more preferentially still from 10 to 24, even better still from 12 to 22 carbon atoms. The aryl group preferably denotes a phenyl or benzyl group. These compounds may be polyoxyalkylenated, in particular polyoxyethylenated, and then preferably comprise from 1 to 50 ethylene oxide units, better still from 2 to 10 ethylene oxide units. It is also possible to use C6-C24 alkyl monoesters and polyglycoside-polycarboxylic acids such as C6-C24 alkyl polyglycoside citrates, C6-C24 alkyl polyglycoside tartrates, and salts thereof.

According to one embodiment, the carboxylic anionic surfactants are chosen from:
C6-C24, or even C12-C20 acylglutamates, such as stearoyl glutamates, and in particular sodium or disodium stearoyl glutamate or cocoyl glutamates, in particular sodium or disodium cocoyl glutamate;
acylsarcosinates, in particular C6-C24 or even C12-C20 acylsarcosinates, such as cocoyl sarcosinates and in particular sodium cocoyl sarcosinate, lauroyl sarcosinates and in particular sodium lauroyl sarcosinate, palmitoylsarcosinates, and in particular sodium palmitoylsarcosinate;
acyllactylates, in particular C12-C28 or even C14-C24 acyllactylates, such as behenoyllactylates, and in particular sodium behenolyl lactylate, (iso) stearoyl lactylates, and in particular sodium (iso) stearoyl lactylate;
acylglycinates, in particular C6-C24 or C12-C20 acylglycinates, such as cocoyl glycinates and in particular sodium cocoyl glycinate;
alkyl(C6-C30) ether carboxylic acids, in particular alkyl(C6-C24) ether carboxylic acids;
alkyl(C6-C30) arylether carboxylic acids, in particular alkyl(C6-C24) arylether carboxylic acids;
alkyl(C6-C30)amidoether carboxylic acids, in particular alkyl(C6-C24)amidoether carboxylic acids;
and mixtures thereof; and in particular in the form of alkali or alkaline earth metal salts, ammonium, or aminoalcohol.

The anionic sulfonate surfactants may be selected from the following compounds: alkylsulfonates, alkylamide sulfonates, alkylaryl sulfonates, C6-C24 alkyl polyglycoside sulfosuccinates, alpha-olefin sulfonates, kerosene sulfonates, alkyl sulfosuccinates, alkyl ether sulfosuccinates, alkylamide sulfosuccinates, alkyl sulfoacetates, N-acyltaurates, acyl isethionates; alkyl sulfolaurates; and salts of these compounds; the alkyl groups of these compounds containing from 6 to 30 carbon atoms, in particular from 12 to 28, even better from 14 to 24, or even from 16 to 22, carbon atoms; the aryl group preferably designating a phenyl or benzyl group; these compounds may be polyoxyalkylenated, in particular polyoxyethylenated and in such case preferably comprising from 1 to 50 ethylene oxide units, better still from 2 to 10 ethylene oxide units.

According to one embodiment, the anionic sulfonate surfactants are selected from: C6-C24, in particular C12-C20 alkyl sulfosuccinates, especially lauryl sulfosuccinates; C6-C24, in particular C12-C20 alkyl ether sulfosuccinates; (C6-C24) acyl isethionates, preferably (C12-C18) acyl isethionates; alpha-olefin sulfonates; and mixtures thereof; and in particular in the form of alkali or alkaline-earth metal, ammonium or aminoalcohol salts.

According to one embodiment, the anionic surfactants may be chosen from C6-C24 alkyl monoesters and polyglycoside-dicarboxylic acids such as alkyl glucoside citrates, alkyl polyglycoside tartrates and alkyl polyglycosidesulfosuccinates, alkylsulfosuccinamates, acyl isethionates and N-acyltaurates, the alkyl or acyl group of all these compounds preferably comprising from 12 to 20 carbon atoms. Another group of anionic surfactants that can be used in the compositions of the present invention is that of acyllactylates, the acyl group of which comprises from 8 to 20 carbon atoms. Furthermore, other examples include alkyl-D-galactosideuronic acids and salts thereof, as well as polyoxyalkylenated (C6-C24)alkyl ether carboxylic acids, polyoxyalkylenated (C6-C24)alkyl(C6-C24) aryl ether carboxylic acids, polyoxyalkylenated (C6-C24)alkyl amidoether carboxylic acids and their salts, in particular those with 2 to 50 ethylene oxide units, and mixtures thereof.

The phosphate anionic surfactants are chosen from: C6-C24 alkyl phosphates, in particular C12-C20 alkyl phosphates; C6-C24 alkyl ether phosphates, in particular C12-C20 alkyl ether phosphates; and mixtures thereof.

The amphoteric or zwitterionic surfactants may be selected from secondary or tertiary or quaternary aliphatic amine derivatives, wherein the aliphatic group is a linear or branched chain comprising from 8 to 22 carbon atoms and containing at least one anionic group such as, for example, a carboxylate, sulfonate, sulfate, phosphate or phosphonate group. Mention may also be made of: (C8-C20)alkyl betaines, for example cocobetaine, sulfobetaines, (C8-C20)alkyl sulfobetaines, (C8-C20)alkyl amidoalkyl(C3-C8) betaines, for example cocamidopropyl betaine; or (C8-C20)alkyl amidoalkyl(C6-C8) sulfobetaines.

Cationic surfactants can be selected from Cetrimonium Chloride marketed under the Microcare Quat CTC 30 reference.

The surfactant(s) is/are present in the composition according to the invention at a content ranging from 0.1% to 40%, preferably from 0.5 to 30%, preferably from 1 to 20%, preferably from 2 to 12% by weight relative to the total weight of the composition.

### —One or More Film-Forming Agent(s)

The film-forming agent can be selected from polymers of natural origin such as hydroxypropyl methylcellulose (HPMC), hydroxypropyl cellulose (HPC), or synthetic polymers such as polyvinyl alcohol (PVA), and/or plasticizers other than polyols, such as polyethylene glycol, triethylcitrate, polysorbate, or else waxes such as Carnauba wax, or hydrogenated castor oil.

Another film-forming agent of plant origin is Beauté by Roquette® ST 720 hydroxypropylated pea starch, sold by ROQUETTE.

The film-forming agent(s) is/are present in the composition according to the invention at a content ranging from 0.1% to 20%, preferably from 0.5 to 15%, preferably from 1 to 12%, by weight relative to the total weight of the composition.

### —One or More Dyestuffs

The cosmetic composition according to the invention may further comprise at least one dyestuff selected from water-soluble or liposoluble dyes, fillers with the effect of coloring and/or opacifying the composition and/or coloring the keratinous materials, preferentially the skin or hair, such as pigments, nacres, lakes (water-soluble dyes adsorbed on an inert mineral support) and mixtures thereof. These dyestuffs may be optionally surface-treated by a hydrophobic agent such as silanes, silicones, fatty acid soaps, C9-15 fluoroalcohol phosphates, acrylate/dimethicone copolymers, C9-15 fluoroalcohol phosphate/silicon mixed copolymers, lecithins, carnauba wax, polyethylene, chitosan and optionally amino acids which could be acylated such as lauroyl lysine, disodium stearoyl glutamate and aluminum acyl glutamate, phytic acid. Another natural colorant is caramel colorant Beauté by Roquette® CC 001, sold by ROQUETTE.

“Pigments” means white or colored, mineral or organic particles intended to color and/or opacify the cosmetic composition.

Among the pigments, mention will be made of mineral or organic, natural or synthetic pigments. Examples of pigments are especially iron, titanium or zinc oxides, and also composite pigments and goniochromatic, pearlescent, interference, photochromic or thermochromic pigments, with this list being non-limiting.

Among the pigments surface-treated with lecithins are UNIPURE WHITE LC981 HLC (INCI: CI 77891 (and) Hydrogenated Lecithin, UNIPURE YELLOW LC182 HLC (INCI: CI 77492 (and) Hydrogenated Lecithin), UNIPURE BLACK LC989 HLC (INCI: CI 77499 (and) Hydrogenated Lecithin, UNIPURE RED LC381 (INCI: CI 77491 (and) Hydrogenated Lecithin) from the company Sensient Cosmetic Technologies.

Among the pigments surface-treated with phytic acid are Unipure White LC 985 PHY (INCI: CI 77891 (and) Phytic Acid (and) Sodium Hydroxide), Unipure Yellow LC 188 PHY (INCI: CI 77492 (and) Phytic Acid (and) Sodium Hydroxide), Unipure Red LC 388 PHY (INCI: CI 77491 (and) Phytic Acid (and) Sodium Hydroxide), Unipure Black LC 998 PHY (INCI: CI 77499 (and) Phytic Acid (and) Sodium Hydroxide), from the company Sensient Cosmetic Technologies.

“Nacres” is intended to mean any iridescent or non-iridescent colored particles which have a color effect by optical interference.

Among nacres, mention will be made of titanium mica covered with metal oxide, such as iron oxide or titanium oxide.

According to one embodiment, the make-up and/or skincare composition according to the invention comprises at least colored particles, preferentially pigments such as iron oxide pigments and/or nacres.

The dyestuff(s) is/are present in the composition according to the invention at a content ranging from 0.05% to 20%, preferably from 1 to 15%, preferably from 0.5 to 12%, by weight relative to the total weight of the composition.

### One or More Fillers(s) or Sensory Powder(s)

This term is intended to mean particles of any form (especially spherical or lamellar), mineral or organic, which are insoluble in the composition. Examples of fillers or sensory powders are talc, boron nitride, starch such as granular starches by Beauté by Roquette® ST 005 and Beauté by Roquette® ST 012 sold by ROQUETTE, polyamides, silicone resins, silicone elastomer powders and acrylic polymer powders, in particular powders of poly(methyl methacrylate) or powders of styrene acrylate copolymer (Sunsphere Powders by Dow).

The filler(s) is/are present in the composition according to the invention at a content ranging from 0.5% to 20%, preferably from 1 to 15%, preferably from 3 to 12%, by weight relative to the total weight of the composition.

### One or More Sensory Agents

Sensory agents can be used to modify the sensory profile of the cosmetic or dermatological composition, making it more pleasant to use on the skin. Sensory agents can be selected from modified starches such as carboxymethylated starches, for example, Roquette's Beauté by Roquette® ST 118, silicas, or talcs, or mixtures thereof. These sensory agents provide a soft touch for solid compositions, or a cushioning effect for compositions in liquid or gel form.

The sensory agent(s) is/are present in the composition according to the invention at a content ranging from 0.1% to 20%, preferably from 1 to 15%, preferably from 3 to 12%, by weight relative to the total weight of the composition.

### —One or More Active Ingredients

In addition to hyperbranched dextrins, the cosmetic composition may comprise one or more active ingredients.

These active ingredients may be of a wide range of chemical types, as the applicant has found that hyperbranched dextrin, preferentially hyperbranched and hydrogenated dextrin, is fairly inert, with low reactivity and excellent compatibility with most active ingredients used in dermatological compositions/topical formulations. These active ingredients may be selected from moisturizing, emollient, film-forming, barrier, anti-pollution, tensing, anti-aging, anti-oxidant, exfoliating, collagen stimulating, anti-acne, sebum reducing, blood circulation stimulating, refreshing, antibacterial, anti-fungal, anti-inflammatory, soothing, anti-irritant, healing, slimming, pigmenting, coloring, mattifying and perfuming agents.

Among the soothing agents, those extracted from plants are preferentially used. C-glycosides such as those described in FR2902998 are particularly suitable as soothing agents.

Preferentially, hyperbranched dextrins may be combined with another anti-inflammatory cosmetic active ingredient or with a soothing active ingredient. Active ingredients include those described in the following table:

TABLE 1

| | | | |
|---|---|---|---|
| Anti-aging/Anti- | Plant ceramides | Triticum vulgare extract | EUK |
| inflammatory | Blackcurrant | Ribes nigrum extract | Northstar Lipids |
| | seed oil | | (UK) |
| | | | Ltd |
| | Borage seed oil | Borago officinalis extract | Loncopan SA |
| | Camelina oil | Camelina sativa extract | Northstar Lipids |
| | | | (UK) Ltd |
| | Echium seed oil | Echium plantagineum | Seatons Ltd |
| | | extract | |
| | Echium seed oil | Echium plantagineum | Bioriginal Food & |
| | | extract | Science Corp. |
| | Euphoryl | Extract of Plukenetia | Cognis France |
| | Omega ™ 3- LS | volubilis (and) Schinus | BASF Div. |
| | | Terebinthifolius | Laboratoires |
| | | | Serobiologiques |
| | Linseed oil | Linum usitatissimum | Kerfoot Group |
| | | extract | Ltd |
| | Incromega | Glycerides, fish oils | Croda |
| | EPA ™ (Omega | | International Pic |
| | EPA) | | |
| | Marine lipid oil | Perna canaliculus | Pharmalink |
| | | extract | International Ltd |
| | Sea buckthorn | Hippophae rhamnoides | Green Health |
| | extract | extract | Botanical |
| | | | Products Co. Ltd |
| Anti- | Boswellia | Boswellia serrata extract | EUK |
| inflammatory | Boswellin ® CG | Boswellia serrata extract | Sabinsa Corp |
| | Devil's claw | Harpagophytum | Advanced |
| | extract | procumbens extract | Phyto Trading CC |
| | Herbalia ® | Ruscus aculeatus | Cognis France |
| | Butcher's | extract | BASF Div. |
| | Broom | | Laboratoires |
| | | | Serobiologiques |
| Anti- | Evodia | Evodia rutaecarpa | In-house |
| inflammatory/NF- | rutaecarpa | extract | extraction |
| kB Inhibitor | extract | | |
| | 5-Loxing ® | 3-o-acetyl-11-keto-beta- | Laila |
| | | boswellic acid | Nutraceuticals/PL |
| | | | Thomas & Co. |
| | | | Inc |
| | MAXnolia | Extract from the bark of | Mibelle AG |
| | | Magnolia officinalis, Vitis | Biochemistry |
| | | vinfera. Tocopherol, | |
| | | Lecithin | |
| Anti-irritant | Herbalia ® | Urtica dioica extract | Cognis France |
| | Nettle | | BASF Div. |
| | | | Laboratoires |
| | | | Serobiologiques |
| | Capsaicin | Capsicum annum extract | Sabinsa Corp |
| | OC Oat Oil | Avena sativa extract | Ceapro Inc |

The active ingredient(s) is/are present in the composition according to the invention at a content ranging from 0.05% to 20%, preferably from 0.1 to 10%, preferably from 0.5 to 6%, by weight relative to the total weight of the composition.

Dermatological compositions according to the invention are particularly suitable for intolerant and/or irritable skin and sensitive scalps

Irritable or intolerant skin has been defined in patent FR2918886.

Intolerant skin is skin that reacts with sensations of heat, tightness, tingling and/or redness to various factors, such as the application of cosmetic or dermatological products. In general, these signs are associated with erythema and hyperseborrheic or acneic skin, or even rosacea, with or without dry patches.

Irritable skin is skin that reacts with pruritus, in other words, itching or tingling, to various factors such as the environment, emotions, food, wind, rubbing, razors, hard water with a high lime content, temperature variations or wool. Most often, skin irritability translates into visible signs such as skin redness, a feeling of hot skin or scalp, or even a feeling of pain.

“Sensitive scalp” is understood to mean scalps for which the sensation of itching and/or tingling and/or heat is essentially triggered by local factors such as rubbing, soap, surfactants, hard water with a high lime content, shampoos or lotions. These sensations are also sometimes triggered by factors such as the environment, emotions and/or food. Scalp erythema, hyperseborrhea and dandruff are frequently associated with the above signs.

### EXAMPLES

### Example 1: In-Vitro Determination of the Effect of Hyperbranched and Hydrogenated Dextrins on Skin Inflammation in a 2D Reconstructed Human Epidermis Model

The aim of the study was to determine the effect of hyperbranched and hydrogenated dextrins on biomarkers of inflammation using a two-dimensional (2D) human epidermis reconstructed model after inflammatory pre-treatment. In this example, the hyperbranched and hydrogenated dextrin Nutriose® HM 06 sold by the Applicant has been used.

### Materials and Methods

### Reconstructed Human Epidermis Model

In vitro reconstructed human skin equivalents closely mimic normal human skin. Since these models reproduce, to a large extent, the barrier function properties of normal human skin, they can be used to screen for potential skin irritants. The model used is a two-dimensional reconstruction of the human epidermis, adapted from the method by Poumay et al., 2004 (Poumay, Y., Dupont, F., Marcoux, S., Leclerk-Smekens, M., Herin, M. And Coquette, A. (2004) A simple reconstructed human epidermis: preparation of the culture model and utilization in vitro studies. Arch. Dermatol. Res. 296:203-211). This model comprises a monolayer of normal human epidermal keratinocytes (NHEK, one of the main cell types of the skin), which have been cultured on inert polycarbonate inserts.

Test substances can be applied locally, directly to the epidermal surface, and their irritant potential and/or efficacy can be assessed using a number of parameters, including expression levels of key cellular markers of inflammation.

### Tissue Exposure to Samples

The epidermises were treated with a 0.5% aqueous solution of Sodium Lauryl Sulfate (SLS) for 6 hours to create inflammation of the reconstructed epidermis, then the surface of the epidermises was rinsed with 500 μL of PBS. 20 μL of different concentrations of hydrogenated and highly branched dextrins (10 and 5% in ultrapure water) and 0.05% Betamethasone cream (Biogaran cream, a corticoid with an anti-inflammatory role) were applied topically to the epidermis for 20 hours at 37° C. No application was made for the SLS control within 20 hours of rinsing with PBS.

Under the same conditions, the negative control (UTC) followed the various technical steps without the application of SLS or sample. Sodium lauryl sulfate (SLS, 0.5% in water) is used as a positive control for epidermal inflammation.

### MTT Cell Proliferation Test

Irritating chemical compounds are able to penetrate the stratum corneum and are cytotoxic to cells in the underlying layers. Cell viability was shown to correlate directly with the irritant potential of the chemical compounds. Cell viability is measured upon metabolism, by mitochondrial succinic dehydrogenase, of the vital dye MTT [3-(4,5-dimethylthiazol-2-yl)-2,5-bromide diphenyltetrazolium, thiazolyl blue; EINECS number 206-069-5, CAS number 298-93-1)], to a blue formazan salt which is measured quantitatively after its extraction from tissues.

The colored solution is measured spectrophotometrically at 570 nm using the Molecular Devices Vmax microplate reader, and absorbance values are recorded using the SOFTmax PRO v4.7.1 software. Statistical analysis is performed using GraphPad Prism v5.03 software.

According to the results of this test,
If viability is less than 50%, then the ingredient is irritant
If viability is greater than 50%, then the ingredient is:
mild irritant if it activates biomarkers of inflammation;
non-irritant if it does not activate biomarkers of inflammation.

### Inflammation Marker Assays

Interleukins IL-1α and IL-8 and tumor necrosis factor-α (TNF-α) are measured to determine whether or not treatment has induced an inflammatory response in the treated tissue.

For this, cell supernatants are collected after the required treatment time and commercially available ELISA kits are used to quantitatively determine the presence of inflammatory markers.

TABLE 2

| Assayed | ELISA kit used |
|---|---|
| marker | Supplier Reference |
| Interleukin-1 | Raybiotech 126 ELH- |
| alpha | IL1 a-1 |
| Interleukin-8 | Raybiotech 126 ELH- |
| | IL8-1 |
| ÎNF-alpha | Raybiotech 126 ELH- |
| | TNFa-1 |

These ELISA kits are designed for quantitative measurement of the aforementioned markers, particularly in culture supernatants using specific human antibodies. Samples and a range of standards are deposited in duplicate in each well (96-well plate). The targeted marker present in a sample is bound by the antibody immobilized in the bottom of the well. The wells are washed and a biotinylated antibody is added. After washing, HRP-streptavidin is deposited in the wells. These are washed again, a solution of TMB substrate is added to the wells and the color develops in proportion to the amount of bound marker. A developer solution changes the color from blue to yellow, and its intensity is measured at 450 nm using the Molecular Devices Vmax microplate reader. Absorbance values are recorded and analyzed using SOFTmax PRO v4.7.1 software. The inflammatory response is interpreted as a sample-to-untreated ratio greater than or equal to 2.

### Semi-Quantitative Fluorescence Assay of Cytokines

In order to obtain a more complete profile of the anti-inflammatory effect induced by hydrogenated and highly branched dextrins, a panel of cytokines was assayed on slide by semi-quantitative assay. Cell culture supernatants collected after epidermal treatment are tested using the Human Cytokine Array G3 kit (RayBiotech).

On a glass slide containing the different specific antibodies, supernatants are deposited at a rate of 100 μL per well. A cocktail of biotin-coupled secondary antibodies is then added. With the addition of streptavidin, signal intensities are detected by fluorescence. Each step is followed by several washes.

### Results

### MTT Cell Proliferation Test

Cytotoxicity of samples was tested using the epidermis model and is expressed as a percentage of untreated control (UTC) tissue.

TABLE 3

| Description of the sample | Viability %* | SD %** |
|---|---|---|
| Untreated control | 100.0 | 0.79 |
| | Average OD | |
| | obtained: 2.311 | |
| 2% SLS positive control | 0.9 | 0.35 |
| 0.5% SLS positive control (6 hours) | 82.1 | 4.09 |
| 0.5% SLS (6 hours) + | 77.1 | 2.88 |
| Betamethasone cream | | |
| Biogaran (0.05% Betamethasone) | | |
| 0.5% SLS (6 hours) + 10% | 79.5 | 3.96 |
| “Nutriose ® HM 06” hyperbranched | | |
| dextrins | | |
| 0.5% SLS (6 hours) + 5% “Nutriose ® | 78.2 | 3.62 |
| HM 06” hyperbranched dextrins | | |

*Mean of 3 replicates;
**SD: standard deviation

There was a significant difference in cell viability between the untreated control and the treated areas, demonstrating the impact of inflammatory treatment with SLS.

After 20 hours, cell viability in the presence of 0.5% SLS and Nutriose HM 06 hyperbranched dextrin was over 50%: thus Nutriose HM 06 hyperbranched dextrin is not an acute irritant for the epidermis. As expected, the same was true for the sample treated with 0.5% SLS and 0.05% betamethasone. Cell viability measured in the presence of 0.5% SLS and Nutriose® HM 06 hyperbranched dextrin was as good as in the presence of 0.5% SLS and betamethasone. As this cell viability test established that Nutriose HM 06 hyperbranched dextrin is not an acute irritant, tests on the expression of inflammatory markers could be carried out to determine whether this dextrin could be qualified as either a mild irritant, in the case where there would be no reduction in inflammatory marker expression, or an anti-irritant, in the case where the expression of at least one major inflammatory marker would be reduced.

### Test on Inflammatory Markers IL-1α, IL-8 and TNF-α

TABLE 4

| | Sample:Untreated ratios | |
|---|---|---|
| | (assayed quantity) | |
| Description of the sample | IL-1a | IL-8 |
| Untreated control | 1.0 | 1.0 |
| | (27 pg/mL) | (140 pg/mL) |
| 0.5% SLS positive control (6 hours) | 2.3 | 3.2 |
| | (61 pg/mL) | (453 pg/mL) |
| 0.5% SLS (6 hours) + Betamethasone | | |
| cream | 1.5 | 3.6 |
| Biogaran (0.05% Betamethasone) | (41 pg/mL) | (511 pg/mL) |
| 0.5% SLS (6 hours) + 10% “Nutriose ® | 1.6 | 3.5 |
| HM 06” hyperbranched dextrins | (44 pg/mL) | (488 pg/mL) |
| 0.5% SLS (6 hours) + 5% “Nutriose ® | 1.7 | 3.6 |
| HM 06” hyperbranched dextrins | (46 pg/mL) | (500 pg/mL) |

Inflammatory marker concentrations were averaged from two replicates of n=3 epidermis in an ELISA test (except for IL-8, n=2). The ratios (marker concentration in the presence of the samples/marker concentration of the untreated control) were calculated for each formulation tested.

A ratio greater than or equal to 2 reflects overexpression of the inflammatory marker while a ratio less than 2 indicates non-expression of the inflammatory marker.

The hyperbranched dextrin “Nutriose® HM 06” according to the invention significantly reduces IL-1α expression (by 25 and 28%). Betamethasone also significantly reduces IL-1α expression (by 33%). Thus, no significant difference was observed between the compound according to the invention and betamethasone, a positive control testifying to the anti-inflammatory effect of the compound according to the invention.

None of the concentrations of hydrogenated and highly branched dextrins decreased interleukin IL-8 expression after epidermal inflammation, nor did betamethasone cream have any effect on the IL-8 ratio. TNF-α expression was not high enough to be detected according to the ELISA kit calibration curve.

### Test on the 4 Inflammatory Markers IL-1α, IL-1β, RANTES and VEGE

Following the results obtained by ELISA testing on the inflammatory marker IL1-α, the effect of the concentration of the hyperbranched dextrin “Nutriose® HM 06” at 5% was evaluated after inflammation of the reconstructed epidermis with 5% SLS.

TABLE 5

| | Sample/Untreated ratios | | | |
|---|---|---|---|---|
| Description of the sample | IL-1α | IL-1β | RANTES | VEGF |
| Untreated control | 1.0 | 1.0 | 1.0 | 1.0 |
| 0.5% SLS positive control (6 hours) | 2.3 | 0.8 | 6.7 | 1.9 |
| 0.5% SLS (6 hours) + 5% “Nutriose ® HM | 1.3 | 0.4 | 5.7 | 1.6 |
| 06” hyperbranched dextrins | | | | |

The ratios (fluorescent signal intensity of the marker in the presence of the samples/fluorescent signal intensity of the marker in the untreated control) were calculated for each treatment from n=3 epidermal culture supernatants (except for the untreated control, n=2).

A ratio >1.5 reflects overexpression of the inflammatory marker.

A ratio <0.65 indicates underexpression of the inflammatory marker.

According to the supplier's criteria, signals are usable when they are greater than the average of the background noise+2SD. Overexpression or underexpression of 5 markers was observed with 0.5% SLS inflammation. A decrease in the expression of these markers was also observed with the addition of the “Nutriose® HM 06” hyperbranched dextrin-based treatment.

Thus, “Nutriose® HM 06” hyperbranched dextrin reduces:
the expression of IL-1α (vs. SLS alone) and passes under the critical limit of 1.5
expression of IL-1β (vs. SLS alone) to below 0.65
expression of IL-15 (compared with the untreated control) to below 0.65
VEGF expression (compared with SLS alone), bringing it closer to the critical threshold of 1.5
RANTES expression (vs. SLS alone).

Nutriose HM 06 hyperbranched dextrin therefore reduces the expression of major markers of inflammation (IL-1 alpha and IL-1 beta), and can therefore be described as an anti-irritant. Said dextrin has anti-inflammatory properties.

### Example 2: In-Vitro Determination of the Effect of Hyperbranched Dextrins on Membrane Integrity in a 3D Reconstructed Human Epidermis Model

The aim of the study was to assess the membrane integrity of skin cells in a three-dimensional (3D) reconstructed human epidermis model, by measuring the amount of the enzyme lactate dehydrogenase (LDH) in the culture supernatant, using colorimetry. LDH is an enzyme that is released in the event of cell death. The absence of LDH secretion is a sign that the integrity of epidermal cell membranes is preserved.

### Protocol

Samples of 3D reconstructed human epidermis were immersed in an aqueous solution containing 0.5% by weight of sodium lauryl sulfate, for 6 hours. The solution was then removed, and the samples immersed in different solutions for 20 hours: 1 sample in a 5% wt. aqueous solution of Nutriose® HM 06, another sample in a 0.5% wt. aqueous solution of bisabolol, and another sample in a 0.5% wt. aqueous solution of betamethasone. The amount of LDH secreted into the supernatant was then determined colorimetrically.

### Results

TABLE 6

| | Membrane |
|---|---|
| Description of the sample | integrity (%) |
| Untreated control | 100 |
| 0.5% SLS (6 hours) + 0.5% bisabolol | 16.8 |
| (20 hours) | |
| 0.5% SLS (6 hours) + 5% “Nutriose ® | 104.5 |
| HM 06” hyperbranched dextrins | |
| (20 hours) | |
| 0.5% SLS (6 hours) + 0.5% | 110.7 |
| betamethasone (20 hours) | |

Roquette's Nutriose® HM 06 hyperbranched dextrin maintains the integrity of reconstructed human epidermal cell membranes as effectively as betamethasone.

### Example 2: Clinical Study of Shaving-Induced Transepidermal Water Loss

The aim of the study is to determine the effect of a hyperbranched dextrin, sold under the reference Nutriose® HM 06 by Roquette, on transepidermal water loss (or TEWL) induced by shaving facial skin through topical application of said dextrin in the form of a lotion (aftershave), in a clinical study on volunteers.

### Volunteers and Products

This non-invasive, single-center study was carried out on sixty male volunteers aged between 30 and 55, with sensitive skin (based on self-reporting) and routinely using mechanical razors and applying aftershave. The inclusion criteria for volunteers are shown in the table.

TABLE 7

| Criteria | |
|---|---|
| Gender | Male |
| Phototype | I to III |
| Age | From 30 to 55 (divided into 3 |
| | homogeneous average age |
| | groups) |
| Sensitive skin | Yes, on the basis of the |
| | volunteer's declaration |
| Regular razor type | Mechanical razor |
| Application of aftershave | regular |

Volunteers were divided into three homogeneous groups of 20 volunteers, and each group received a batch of double-blade razors, “commercial reference” shaving cream, and aftershave lotion according to the table below.

### Composition of Aftershave Creams

TABLE 8

| | | Volunteer group No. | | |
|---|---|---|---|---|
| | | | | 3 |
| | | | | Lotion |
| | | 1 | 2 | according |
| | | Placebo | Reference | to the |
| Ingredient/Supplier | INCI | lotion | lotion | invention |
| Aqua | | 96.8 | 96.3 | 91.8 |
| Dragosantol ® 100 | Bisabolol | 0 | 0.5 | 0 |
| Nutriose ® HMC06 | Not available | 0 | 0 | 5 |
| Cosmedia ® SP | Sodium | 0.2 | 0.2 | 0.2 |
| | polyacrylate | | | |
| Caprylis/Labrafac | Caprylic/capric | 2 | 2 | 2 |
| CC | triglycerides | | | |
| Microcare ® PHC | Phenoxyethanol | 1.00 | 1.00 | 1.00 |
| | and glycerin | | | |
| Citric acid | | Qs pH 6 | Qs pH 6 | Qs pH 6 |

### Equipment

The faces of the volunteers are assessed by measuring trans epidermal water loss (TEWL). This measurement allows the barrier function of the skin to be assessed: the value of water loss is inversely proportional to the barrier function of the skin. Transepidermal water evaporation (expressed in g/h/m2) was measured on a Tewameter TM300® from Courage & Khazaka electronics, using the open room diffusion technique. Around twenty successive measurements are taken on the surface of the face subjected to shaving, and an average value is used.

### Study Procedure

Volunteers replaced their usual razor and shaving cream with the study razor and shaving cream three days before the start of the study. The study then began by assessing the faces of the volunteers in the laboratory after shaving and applying aftershave lotion in the laboratory (“day 0” measurement). The measurement was repeated 10 minutes after the “day 0” measurement.

Between day 0 and day 13, volunteers performed their daily shave at home using a new razor for each shave and the study's shaving cream and aftershave lotion. On day 14, the volunteers returned to the laboratory for a facial assessment following the same protocol as on day 0, in other words, an assessment after shaving followed by the application of aftershave lotion in the laboratory, followed by a second measurement 10 minutes after the first.

Between day 14 and 27, volunteers repeated the same protocol as between days 0 and 13. Then, on day 28, they returned to the laboratory for a final facial assessment using the same protocol as on day 0 and day 14: that is, an assessment on day 28 after shaving and application of aftershave lotion, followed by a second measurement 10 minutes after the first.

### Results

TABLE 9

| TEWL | Group 1 | Group 2 | Group 3 |
|---|---|---|---|
| assessment | placebo | reference | invention |
| Day 0 | 21.74 | 22.07 | 22.59 |
| Day 0 + | 19.51 | 16.88 | 18.83 |
| 10 minutes | | | |
| Day 14 | 21.38 | 22.58 | 22.40 |
| Day 14 + | 19.65 | 18.98 | 19.39 |
| minutes | | | |
| Day 28 | 19.98 | 20.77 | 24.19 |
| Day 28 + | 18.99 | 18.02 | 19.58 |
| 10 minutes | | | |

TABLE 10

| Variation | | | |
|---|---|---|---|
| (Day x + | | | |
| 10 minutes) − | Group 1 | Group 2 | Group 3 |
| Day x | placebo | reference | invention |
| Day 0 | −2.24 | −5.19 | −3.77 |
| Day 14 | −1.73 | −3.60 | −3.01 |
| Day 28 | −0.99 | −2.75 | −4.62 |

The study thus showed that application of an aftershave lotion comprising the hyperbranched and hydrogenated dextrin according to the invention, Nutriose HMC 6, reduced intracellular water loss compared with placebo lotion from day 0 and on days 14 and 28. It also reduced water loss on day 14 to the same extent as the reference product, and reduced water loss on day 28 to a greater extent than the reference product.

### Example 3: Clinical Study of Transepidermal Water Loss (TEWL) and Redness Induced by Sodium Lauryl Sulfate

The aim of the study was to determine the effect of a hyperbranched dextrin, sold under the reference Nutriose® HM 06 by Roquette, on the redness created by irritation of the skin of the inner forearm by a patch-test with 10% SLS, during a clinical test on volunteers.

### Volunteers and Products

The volunteers were the same as for the clinical study in Example 2 (shaving study), divided into the same groups, with the same products (Table 6 in Example 2).

### Materials

Skin color was assessed colorimetrically using a Minolta CM2600D™ spectrophotometer. Color measurements were made using the following parameters: standard 10° field of view, D65 illumination corresponding to daylight, measurement on 8 mm square areas, L*a*b* color space used, SCI method (specular included) to avoid surface conditions.

The vertical dimension L* represents luminosity (or luminance), with values ranging from 0 (black) to 100 (white). Parameter a* determines a range of 600 levels on the red axis (+299 positive values) through gray (value 0) to green (−300 negative values). Parameter b* determines a range of 600 levels on the yellow axis (+299 positive values) through gray (value 0) to blue (−300 negative values). For this study, the parameter a*, representing skin redness, was determined.

### Study Protocol

At the start of the study (day 0), measurements of skin color and TEWL were taken on defined areas on both forearms of volunteers. 10% SLS patches were then placed on the two defined areas on the forearms of each volunteer. Volunteers kept the patches on for 20 hours, and removed them 4 hours before the Day 1 measurement, so that on Day 1, color and TEWL measurements were taken on both forearms of all volunteers. Immediately after the Day 1 measurement, aftershave lotion was applied to the SLS patch-treated area on the right forearm, then color and TEWL were measured 5, 10 and 30 minutes after this application. The left forearm received no treatment.

From day 1 to day 6, volunteers applied aftershave lotion to the SLS-treated area of the right forearm every evening. The left forearm received no treatment. On days 2, 3, 4 and 7, the color and TEWL of the area treated with aftershave lotion and the control area were measured.

### Results

TABLE 11

| Param- | Placebo group | | Reference group | | Invention group | |
|---|---|---|---|---|---|---|
| eter a* | Treated | Untreated | Treated | Untreated | Treated | Untreated |
| Day 0 | 4.56 | 5.05 | 4.46 | 4.88 | 4.29 | 4.81 |
| Day 1 | 6.17 | 6.12 | 6.92 | 6.45 | 6.41 | 6.29 |
| Day 1 + | 6.11 | 6.05 | 6.67 | 6.19 | 5.74 | 6.00 |
| 5 min | | | | | | |
| Day 1 + | 5.89 | 6.07 | 6.80 | 6.09 | 5.49 | 6.02 |
| 10 min | | | | | | |
| Day 1 + | 6.01 | 5.59 | 6.20 | 5.80 | 5.28 | 5.88 |
| 30 min | | | | | | |
| Day 2 | 6.51 | 6.68 | 6.75 | 6.90 | 6.43 | 7.13 |
| Day 3 | 6.54 | 7.05 | 6.95 | 7.30 | 6.66 | 7.37 |
| Day 4 | 6.55 | 6.80 | 6.51 | 6.38 | 6.43 | 6.81 |
| Day 7 | 5.97 | 6.09 | 5.96 | 5.87 | 5.67 | 6.25 |

To compare the changes in redness between the different groups, the variations in redness between the different measurement times were calculated, for example for a treated area of skin:

$$[ Math . 1 ]$$

$$Δ Treated ( ( Day x ) - Day 1 ) = ( {a}^{*} of the treated area on day x ) - ( {a}^{*} of the treated area on day 1 ) ,$$

$$for all three groups .$$

TABLE 12

| | Placebo | Reference | Invention |
|---|---|---|---|
| Variation in parameter a* | group | group | group |
| ΔTreated((Day 1 + 5 min) − | −0.02 | 0.02 | −0.38 |
| Day 1) − ΔUntreated ((Day 1 + | | | |
| 5 min) − Day 1) | | | |
| ΔTreated((Day 1 + 10 min) − | −0.23 | 0.24 | −0.66 |
| Day 1) − ΔUntreated ((Day 1 + | | | |
| 10 min) − Day 1) | | | |
| ΔTreated((Day 1 + 30 min) − | 0.38 | −0.06 | −0.73 |
| Day 1) − ΔUntreated ((Day 1 + | | | |
| 30 min) − Day 1) | | | |
| ΔTreated((Day 2) − Day 1) − | −0.22 | −0.61 | −0.82 |
| ΔUntreated ((Day 2) − Day 1) | | | |
| ΔTreated((Day 3) − Day 1) − | −0.55 | −0.81 | −0.84 |
| ΔUntreated ((Day 3) − Day 1) | | | |
| ΔTreated((Day 4) − Day 1) − | −0.30 | −0.33 | −0.50 |
| ΔUntreated((Day 4) − Day 1) | | | |
| ΔTreated((Day 7) − Day 1) − | −0.17 | −0.37 | −0.70 |
| ΔUntreated((Day 7) − Day 1) | | | |

The table shows the variation in redness over time between treated and untreated areas of skin

Nutriose® HM 06 hyperbranched dextrin significantly reduces SLS-induced redness as early as 10 minutes compared with the placebo and reference groups, and this reduction is maintained for up to 7 days. Reducing skin redness by applying Nutriose HM 06 hyperbranched dextrin showed that this dextrin reduced the skin inflammation caused by the 10% SLS patch.

In addition, this reduction in inflammation was faster than for the reference group, and therefore faster than for the well-known anti-inflammatory Bisabolol.

TABLE 13

| | Placebo | Reference | Invention |
|---|---|---|---|
| Parameter a* | group | group | group |
| Day 0 | 8.88 | 9.35 | 9.42 |
| Day 1 | 19.12 | 23.95 | 24.27 |
| Day 1 + 30 min | 18.78 | 23.17 | 19.21 |
| Day 2 | 20.28 | 23.44 | 23.17 |
| Day 3 | 19.37 | 20.74 | 21.22 |
| Day 4 | 15.68 | 16.38 | 16.82 |
| Day 7 | 15.73 | 14.55 | 14.24 |

The table shows transepidermal water loss in treated areas of the forearm

TABLE 14

| | Placebo | Reference | Invention |
|---|---|---|---|
| Parameter a* | group | group | group |
| (Day 1 + 30 min) − | −0.34 | −0.775 | −5.06 |
| Day 1 | | | |
| Day 2 − Day 1 | 1.16 | −0.505 | −1.095 |
| Day 3 − Day 1 | 0.245 | −3.205 | −3.045 |
| Day 4 − Day 1 | −3.436 | −7.565 | −7.445 |
| Day 7 − Day 1 | −3.39 | −9.4 | −10.025 |

Table 14 shows variations in transepidermal water loss in the forearm treated areas compared with day 1.

Nutriose® HM 06 hyperbranched dextrin significantly reduced transepidermal water loss as early as 30 minutes after application, then progressively increased over the 2-7 day treatment period, to reach a value of water loss reduction greater than that of the placebo group by around 6.6 units (that is, a difference of around 195%). Said hyperbranched dextrin therefore helped to maintain skin hydration by maintaining the skin barrier function.

### Example 4: Cosmetic Product Formulations Comprising a Hydrogenated and Highly Branched Dextrin as Claimed Herein

### “Skin Lover” Cream (SC-079-005)

TABLE 15

| Phase | INCI | Trade name | Supplier | % w/w |
|---|---|---|---|---|
| A1 | Aqua | Demineralized | | 59.93 |
| | | water | | |
| | Not available | Nutriose HM 06 | Roquette | 5.00 |
| A2 | Xanthan gum | Keltrol CG | CP Kelco | 0.30 |
| | Microcrystalline cellulose, | Tabulose SC 611 | Itacel | 1.00 |
| | cellulose gum | | | |
| A3 | Cyclodextrin, sorbitol, | Beauté by | Roquette | 5.00 |
| | polyglyceryl-3- | Roquette DS 146 | | |
| | diisostearate | | | |
| B | Diheptyl succinate, | Lexfeel N350 | Inolex | 20.00 |
| | capryloyl glycerin/sebacic | | | |
| | acid copolymer | | | |
| C | Zea Mays starch | Beauté by | Roquette | 5.00 |
| | | Roquette ST 005 | | |
| D | Gluconic acid, | Beauté by | Roquette | 1.00 |
| | caprylyl/capryl glucoside, | Roquette LS 007 | | |
| | cymbopogon | | | |
| E | Aqua | Demineralized | | 2.00 |
| | | water | | |
| | Potassium sorbate | Microcare KS | Thor | 0.40 |
| F | Fragrance | Delicious apple | Laboratoires | 0.30 |
| | | IPO | L.R.Flavours | |
| | | | & Fragrances | |
| | | | industries | |
| | | | S.p.A | |
| | Aqua and CI 42090 | FD&C Blue 1 | Sensient | 0.07 |
| | | (2% aqueous | | |
| | | solution) | | |

Preparation protocol: at 45° C., Nutriose® HM 06 was added to water and mixed with a deflocculator at 500 rpm until a clear solution was obtained. The ingredients of phase A2 were then mixed together in water with stirring at 500-1000 rpm. Phase A3 was then added to phase A1+A2, and stirred at 1500 rpm for 10 minutes. Separately, phase B was prepared by heating to 45° C., then the liquid phase B was emulsified in phase A1+A2+A3 at 45° C. with 2000-3000 rpm stirring for 15 minutes. It was then cooled to room temperature, and phases C and D were added. We finished by adding E and F, previously dissolved in a little water. The result was a blue emulsion with a Brookfield viscosity of 4500-5500 mPa·s (SP3 mobile, 20 rpm, 20° C.).

### After-Sun Cream

TABLE 16

| Phase | INCI | Trade name | Supplier | % w/w |
|---|---|---|---|---|
| A | Aqua | Demineralized | / | To 100 |
| | | water | | |
| | Sorbitol | Beauté by | Roquette | 5 |
| | | Roquette ® PO 071 | | |
| | Not available | Nutriose HM 06 | Roquette | 5 |
| | Xanthan gum | Xanthan Gum FNC | JBL | 0.3 |
| | | SP- PC | | |
| | Polyglyceryl-10 | Emulpharma Eco10 | Res Pharma | 3 |
| | Laurate | | | |
| B | Hydrogenated | Nat Organic Argan | Naturochim | 2 |
| | Argania Spinosa | Wax | | |
| | Kernel Oil | | | |
| | Butyrospermum | Shea butter | Aroma Zone | 3 |
| | Parkii (Shea) Butter | | | |
| | Jojoba Esters | Acticire MB | Gattefossé | 5 |
| | and Helianthus | | | |
| | Annuus (Sunflower) | | | |
| | Seed Wax (and) | | | |
| | Acacia Decurrens | | | |
| | Flower Wax (and) | | | |
| | Polyglycerin-3 | | | |
| | Argania Spinosa | Deodorized virgin | Cooper | 12 |
| | (argan) Kernel Oil | argan oil | | |
| | Tocopherols (mixed), | Vitamin E | Aroma Zone | 0.2 |
| | helianthus annuus | | | |
| | seed oil | | | |
| C | Aluminium starch | Beauté by | Roquette | 3 |
| | octenylsuccinate | Roquette ® ST 012 | | |
| D | Aqua | Demineralized | / | 5 |
| | | water | | |
| | Potassium sorbate | Potassium sorbate | Cooper | 0.4 |
| | Sodium benzoate | Sodium benzoate | Cooper | 0.4 |

Preparation protocol: phase A and phase B were prepared at 70° C. in two separate beakers. Phase B was then emulsified in phase A under high shear mixing for 10 minutes. This was followed by cooling to 20° C.+/−2° C. Phase C was then added with stirring, followed by phase D. Finally, the pH was adjusted to 6. A white cream was obtained, with a Brookfield viscosity of 3500 mPa·s+/−700 (spindle no. 4 at 20 rpm).

### After-Shave Gel

TABLE 17

| Phase | INCI | Trade name | Supplier | % w/w |
|---|---|---|---|---|
| A | Aqua | Demineralized | / | To 100 |
| | | water | | |
| | Isosorbide | Beauté by | Roquette | 5 |
| | | Roquette ® PO 500 | | |
| | Stach acetate, | Beauté by | Roquette | 3 |
| | hydroxyethylcellulose | Roquette ® DS 112 | | |
| | and xanthan gum | | | |
| | Not available | Nutriose HM 06 | Roquette | 5 |
| B1 | Aqua | Demineralized | / | 5 |
| | | water | | |
| | Potassium sorbate | Potassium sorbate | Cooper | 0.4 |
| B2 | Gluconic acid, | Beauté by | Roquette | 1 |
| | caprylyl/capryl | Roquette ® LS 007 | | |
| | glucoside, | | | |
| | cymbobogon citratus | | | |
| | leaf oil | | | |
| C | Sodium hydroxide | Sodium hydroxide | / | Q.s. |
| | 18% | 18% | | pH 5.5-6 |

Preparation protocol: isosorbide and Nutriose HMC 06 was mixed beforehand in the quantity of water required for phase A, then Beauté by Roquette DS 112 starch was dispersed therein at 80° C. for 30 to 40 minutes. Phases B1 and B2 were then added successively, and the pH adjusted to 6. A translucent gel was obtained, with a Brookfield viscosity of 3500 mPa·s+/−700 (spindle no. 4 at 20 rpm).

### Soothing Oil-In-Water Emulsion

TABLE 18

| Phase | INCI | Trade name | Supplier | % w/w |
|---|---|---|---|---|
| A1 | Aqua | Demineralized | / | 61.93 |
| | | water | | |
| | Not available | Nutriose HM 06 | Roquette | 5.00 |
| A2 | Xanthan gum | Keltrol CG | CP Kelco | 0.30 |
| | Microcrystalline | Tabulose | Roquette | 1.00 |
| | cellulose, cellulose | | | |
| | gum | | | |
| A3 | Cyclodextrin, | Beauté by | Roquette | 5.00 |
| | Sorbitol, Polyglyceryl- | Roquette ® DS 146 | | |
| | 3 diisostearate | | | |
| B | Diheptyl Succinate, | Lexfeel N350 | Inolex | 20.00 |
| | Capryloyl | | | |
| | Glycerin/Sebacic | | | |
| | Acid Copolymer | | | |
| C | Zea Mays Starch | Beauté by | Roquette | 5.00 |
| | | Roquette ® ST 005 | | |
| D | Gluconic acid, | Beauté by | Roquette | 1.00 |
| | caprylyl/capryl | Roquette ® LS 007 | | |
| | glucoside, | | | |
| | cymbopogon citratus | | | |
| | leaf oil | | | |
| E | Potassium sorbate | Microcare KS | Thor | 0.40 |
| F | Perfume | Delicious apple | LR | 0.30 |
| | | IPO | | |
| | Color E 133 | FD&C Blue 1 (2% | | 0.07 |
| | | aq. sol.) | | |

Preparation protocol: Nutriose HMC 06 was added to half the volume of water required for the formulation at 45° C., stirred with a deflocculator at 500 rpm until a clear solution was obtained. Separately, the ingredients of phase A2 were mixed in half the volume of water required for the formulation with stirring at 500-1000 rpm. Phase A1 was then mixed with phase A2, and phase A3 added with stirring at 1500 rpm for 10 minutes. Separately, phase B was prepared by heating to 45° C. Phase B was then emulsified in phase A1+A2+A3 with vigorous stirring at 2000-3000 rpm for 15 minutes at 45° C. It was cooled to 20° C.+/−2° C., then phases C, D, E and F were successively added with 500 rpm stirring, and pH adjusted to 5.3+/−0.2. The result is a blue emulsion with a Brookfield viscosity of 4500-5500 mPa·s (spindle no. 3 at 20 rpm).

Soothing and repairing hand cream

TABLE 19

| Phase | INCI | Trade name | Supplier | % w/w |
|---|---|---|---|---|
| A | Aqua | Water | — | 60.3 |
| | Not available | Nutriose HM 06 | Roquette | 5 |
| B | Starch Acetate, | Beauté by | Roquette | 3 |
| | Starch sodium | Roquette | | |
| | octenylsuccinate, | DS 421 | | |
| | Guargum, Taragum, | | | |
| | Xanthan gum | | | |
| C | Prunus amygdalus | Sweet almond oil | Cooper | 30 |
| | dulcis oil | | | |
| | Fragrance | Nola Fresh Juice | L.R.Flavours | 0.2 |
| | | | & Fragrances | |
| | Benzyl alcohol | Microcare alcohol | Thor | 0.5 |
| | | BNA | | |
| D | Gluconic acid, | Beauté by | Roquette | 1 |
| | caprylyl/capryl | Roquette ® LS 007 | | |
| | glucoside, | | | |
| | cymbopogon citratus | | | |
| | leaf oil | | | |

Preparation protocol: Nutriose HMC 06 was added to the volume of water required for the formulation at 45° C., stirred with a deflocculator at 500 rpm until a clear solution was obtained, then cooled to 20° C.+/−2° C. Beauté by Roquette DS 421 was then dispersed in phase A whilst stirring with a deflocculator at 1000 rpm for approximately 10 minutes to hydrate this ingredient, which was visually observed by its opalescence. Phase C was prepared separately. At room temperature (20° C.+/−2° C.), phase C was added to phase A+B slowly and with stirring using a deflocculator at 2000-3000 rpm, then stirring was maintained for 10 minutes. Finally, phase D was added whilst stirring at 1000-2000 rpm, then adjusted to pH 4.5-4.7. A yellow cream was obtained, with a Brookfield viscosity of 8800 mPa·s (spindle 3 at 20 rpm).

### Soothing Cleansing Cream (TQ-027-001)

TABLE 20

| Phase | INCI | Trade name | Supplier | % w/w |
|---|---|---|---|---|
| A1 | Aqua | Demineralized | — | 28.3 |
| | | Water | | |
| | Not available | Nutriose HM 06 | Roquette | 5 |
| | Xanthan Gum | Keltrol | CP Kelco | 0.7 |
| A2 | Cyclodextrin, Sorbitol, | Beauté by | Roquette | 6 |
| | Polyglyceryl-3 diisostearate | Roquette ® DS | | |
| | | 146 | | |
| B | Helianthus annuus seed oil | Sunflower oil | Cooper | 35 |
| C | Glycerin (and) | Microcare PHC | Thor | 1 |
| | Phenoxyethanol (and) | | | |
| | Chlorphenesin | | | |
| D | Sodium Lauryl ether Sulfate | Texapon NSO UP | BASF | 7 |
| | Coco-glucoside | Pureact Gluco C | Innospec | 5 |
| | Disodium Laureth | Rewopol SB FA | Evonik | 5 |
| | Sulfosuccinate | 30 B | | |
| | Cocamidopropyl Betaine | Dehyton PK45 | BASF | 7 |

### Soothing Hair Conditioning Gel (HC-040-001)

TABLE 21

| Phase | INCI | Trade name | Supplier | % w/w |
|---|---|---|---|---|
| A1 | Aqua | Demineralized | / | 81 |
| | | water | | |
| | Maltitol | Beauté by | Roquette | 5 |
| | | Roquette ® PO | | |
| | | 455 | | |
| | Not available | Nutriose HM 06 | Roquette | 5 |
| A2 | Starch acetate, | Beauté by | Roquette | 5 |
| | Hydroxyethyl-cellulose, | Roquette ® DS | | |
| | Xanthan gum | 112 | | |
| A3 | Cetrimonium Chloride | Microcare Quat | Thor | 3 |
| | | CTC 30 | | |
| B | Phenoxyethanol, | Microcare PHC | Thor | 1 |
| | Chlorphenesin, Glycerin | | | |

### Soothing Hair Conditioning Cream (HC-040-002)

TABLE 22

| | | | | |
|---|---|---|---|---|
| Phase | INCI | Trade name | Supplier | % w/w |

| | | | |
|---|---|---|---|
| A1 | Aqua | Demineralized | / |
| | | water | |
| | Maltitol | Beauté by | Roquette |
| | | Roquette@ PO | |
| | | 455 | |
| | Not available | Nutriose HM 06 | Roquette |
| A2 | Starch acetate, | Beauté by | Roquette |
| | Hydroxyethyl cellulose, | Roquette ® DS | |
| | Xanthan gum | 112 | |
| A3 | Cetrimonium Chloride | Microcare Quat | Thor |
| | | CTC 30 | |
| B | Caprylic/capric triglycerides | Caprylis | Aroma |
| | | | Zone |
| C | Phenoxyethanol, | Microcare PHC | Thor |
| | Chlorphenesin, Glycerin | | |

1. Hyperbranched dextrins for topical use in the prevention or treatment of at least one symptom selected from redness, heat, swelling and pain, wherein the hyperbranched dextrins comprise:
at most 90% of 1,4 glucosidic bonds,
at least 5% of 1,6 glucosidic bonds, relative to the sum of 1,2, 1,3, 1,4 and 1,6 bonds.

2. The hyperbranched dextrins for use according to claim 1, wherein they have:
at least 5% of 1,6 glucosidic bonds relative to the sum of 1,2, 1,3, 1,4 and 1,6 bonds, preferentially at least 10%, more preferentially at least 12%, and most preferentially at least 15%,
at most 70% of 1,4 glucosidic bonds relative to the sum of 1,2, 1,3, 1,4 and 1,6 bonds, preferentially at most 60%, and most preferentially at most 50%,
at least 1% of 1,3 glucosidic bonds relative to the sum of 1,2, 1,3, 1,4 and 1,6 bonds, preferentially at least 5%, more preferentially at least 10%, and most preferentially at least 20%,
at least 1% of 1,2 glucosidic bonds relative to the sum of 1,2, 1,3, 1,4 and 1,6, preferentially at least 5%, more preferentially at least 10%, and most preferentially at least 20%.

3. The hyperbranched dextrins for use according to claim 1, wherein the hyperbranched dextrins have:
from 42 to 50% of 1,4 glucosidic bonds relative to the sum of 1,2, 1,3, 1,4 and 1,6 bonds,
from 5% to 40% of 1,6 glucosidic bonds relative to the sum of 1,2, 1,3, 1,4 and 1,6 bonds,
from 1 to 20% of 1,3 glucosidic bonds relative to the sum of 1,2, 1,3, 1,4 and 1,6 bonds,
from 1 to 20% of 1,2 glucosidic bonds relative to the sum of 1,2, 1,3, 1,4 and 1,6 bonds.

4. The hyperbranched dextrins for use according to claim 1, wherein the dextrins are hyperbranched hydrogenated dextrins.

5. The hyperbranched dextrins for use according to claim 1, wherein the symptom is located in at least one epithelial tissue selected from keratinized multi-layered squamous epithelium, non-keratinized multi-layered squamous epithelium, stratified columnar epithelium, ciliated or non-ciliated pseudostratified columnar epithelium.

6. The hyperbranched dextrins for use according to claim 1 for the prevention or treatment of the symptoms of skin inflammation.

7. The hyperbranched dextrins for use according to claim 1, for the prevention or treatment of the symptoms of mucosal inflammation.

8. The hyperbranched dextrins for use according to claim 6, wherein the skin inflammation is induced by skin irritation, skin corrosion, skin inflammation of endogenous origin, or a combination of these causes.

9. The hyperbranched dextrins for use according to claim 7, wherein the inflammation of the mucous membranes is induced by ingredients contained in cosmetic, dermocosmetic, dermatological, pharmaceutical, medicinal, veterinary or domestic compositions or by mechanical aggression.

10. The hyperbranched dextrins for use according to claim 1 wherein the at least one symptom or skin irritation or skin corrosion or skin inflammation is induced by at least one of the conditions selected from infectious agents, chemicals, ingredients contained in cosmetic, dermatological, pharmaceutical, veterinary, detergent or domestic compositions, mechanical aggressions, environmental factors, thermal aggressions, food and beverages.

11. The hyperbranched dextrins for use according to claim 1, wherein the skin irritations are selected from cumulative irritant contact dermatitis, acute irritant contact dermatitis and phototoxic dermatitis.

12. The hyperbranched dextrins for use according to claim 6, wherein the skin inflammation is induced by a dermatological pathology involving an inflammatory process.

13. The hyperbranched dextrins for use according to claim 12, wherein the pathology is selected from erythema, psoriasis, cutaneous atopy, atopic dermatitis, immediate hypersensitivity type allergic reactions, delayed hypersensitivity type allergic reactions, inflammatory hyperpigmentation, immune dermatoses, actinic elastosis, alopecia areata, vitiligo, systemic lupus erythematosus, pemphigus vulgaris, dystrophic epidermolysis bullosa and autoimmune canities, erythemas, etc.

14. The hyperbranched dextrins for use according to claim 7 wherein mucosal inflammation is induced by a pathology involving an inflammatory process.

15. A use of hyperbranched dextrins to prevent or reduce or eliminate the irritant effect of ingredients contained in cosmetic or dermatological, medicinal, veterinary, detergent or domestic compositions, wherein the hyperbranched dextrins comprise:
at most 90% of 1,4 glucosidic bonds,
at least 5% of 1,6 glucosidic bonds,
relative to the sum of 1,2, 1,3, 1,4 and 1,6 bonds.

16-21. (canceled)
